(19) United States
(12) Patent Application Publication
(10) Pub. No.: US 2019/0100811 A1
(43) Pub. Date: Apr. 4, 2019

(54) PHAGE-BASED DETECTION METHOD FOR ANTIMICROBIAL SUSCEPTIBILITY TESTING AND IDENTIFICATION OF BACTERIAL SPECIES

(71) Applicants: QUIDEL CORPORATION, San Diego, CA (US); GUILD ASSOCIATES, INC., Dublin, OH (US)

(72) Inventors: Alexander Solomon BELENKY, San Diego, CA (US); Werner KROLL, Wayland, MA (US); Todd Denison PACK, San Diego, CA (US); David A. SCHOFIELD, Hollywood, SC (US)

(21) Appl. No.: 16/149,019

(22) Filed: Oct. 1, 2018

Related U.S. Application Data
(60) Provisional application No. 62/566,864, filed on Oct. 2, 2017.

(51) Int. Cl. C12Q 1/6897; C12Q 1/04

(52) U.S. Cl. CPC C12Q 1/6897; G01N 2800/26; C12Q 1/04

(57) ABSTRACT

Methods for determining bacterial identity and susceptibility or resistance to antibiotic or antimicrobial agents are provided. In one embodiment, the bacteria is cultured in the presence or absence or the antibiotic agent to generate a plurality of primary cultures, which are then cultured in the presence or absence of transforming phages to generate a first secondary culture that comprise transformed bacteria that have been treated with the antibiotic agent and a second secondary culture that comprises transformed bacteria that have not been treated with the antibiotic agent. The recombinant phages are specific to the bacteria and comprise a heterologous marker. The susceptibility or resistance of the bacteria to the antibiotic or antimicrobial agent is determined by comparing a level or activity of the marker in the first and second secondary cultures.

### CROSS-REFERENCE TO RELATED APPLICATIONS

This application claims the benefit of U.S. Provisional Application No. 62/566,864, filed Oct. 2, 2017, incorporated herein by reference in its entirety

### TECHNICAL FIELD

The subject matter described herein relates methods for identifying bacterial pathogens and defining the susceptibility of bacteria to test agents and to methods for the determining whether a target bacterial species is resistant to one or more antimicrobial agents. Further embodiments are directed to methods for screening new test compounds for their antimicrobial or probiotic activity, including, detecting the presence of such agents in biological samples, including clinical samples, and food and environmental samples.

### BACKGROUND

Since the first practical use of the antibiotic penicillin, many other antibacterial agents have been developed, and antibacterial therapy has greatly contributed to the advancement of modern medicine and the extension of the average lifespan. However, pathogenic bacteria have acquired resistance to a majority of the antibacterial agents, thereby compromising the overall effectiveness of antibacterial therapy while also presenting new public health problems. In particular, methicillin-resistant Staphylococcus aureus (MRSA), which demonstrates resistance to β-lactam antibacterial agents, is a highly resistant pathogen. It is directly associated with nearly 94,000 new hospitalizations annually, leading to roughly 19,000 deaths/year in the U.S. alone (Voss et al., International Journal of Antimicrobial Agents, 5: 101-106, 1995; McGeer et al., LPTP Newsletter, 190:1-4, 1996; CDC MRSA tracking). Partly owing to increased use of antibiotics in animal husbandry and hospitals, new strains of multi-drug resistant bacteria are also emerging at an alarming rate. For instance, there have been reports of vancomycin intermediate S. aureus (VISA) infections in patients being treated with vancomycin for MRSA infections (Hiramatsu et al., JAntimicrob Chemother, 40(1), 135-6, 1997; Perichon et al., Antimicrob Agents Chemother., 53(11):4580-7, 2009). Indeed, some strains have become resistant to practically all of the commonly available agents. A notorious case is the Mu50 strain of MRSA, which is also resistant to aminoglycosides, macrolides, tetracycline, chloramphenicol, and lincosamides (Hiramatsu et al., supra). Multi-drug resistant Mycobacterium tuberculosis, which is resistant to isoniazid and rifampicin, has also been identified (Dalton et al., Lancet, 380:1406-17, 2012).

Food-borne bacterial diseases, especially those triggered by drug-resistant bacteria, also pose a significant threat to human health. A microbiological study analyzing 150 food samples comprising vegetable salad, raw egg-surface, raw chicken, unpasteurized milk, and raw meat for E. coli revealed that the highest percentages of drug-resistant E. coli isolates were detected in raw chicken (23.3%) followed by vegetable salad (20%), raw meat (13.3%), raw egg-surface (10%) and unpasteurized milk (6.7%). The overall incidence of drug resistant E. coli was 14.7% (Rasheed et al., Rev Inst Med Trop Sao Paulo, 56(4):341-346, 2014). The study further highlights the threat posed by the ability of drug-resistant E. coli to transfer drug resistance genes to other species, e.g., Klebsiella sp.

Increasing scientific evidence points to how bacteria are evolving defense systems to protect against five major classes of antibacterial drugs that are presently in use. These drugs are broadly categorized as β-lactams, β-lactamase inhibitors, cephalosporins, quinolones, aminoglycosides, tetracyclines/glycylcyclins and polymyxins. The limitations of each agent, especially, when used in singularity, are outlined below.

β-lactams are a large class of broad-spectrum drugs that are the main treatment for Gram-negative infections. The subclasses of β-lactam drugs range from narrow-spectrum (penicillin) to broad-spectrum (carbapenem). Gram-negative bacteria have developed several pathways to β-lactam resistance. Perhaps the most concerning mechanism involves evolution of β-lactamases, enzymes that destroy the β-lactam antibiotics. Some β-lactamases destroy narrow spectrum drugs (e.g., only active against penicillin) while newer β-lactamases (e.g., carbapenemases found in carbapenem resistant Enterobacteriaceae or CRE) are capable of neutralizing all β-lactam antibiotics.

β-lactamase inhibitors are still active against Gram-negative bacteria that have β-lactamases with limited activity for destroying β-lactam antibiotics. Bacteria that are resistant to extended-spectrum cephalosporins and carbapenems are usually resistant to these drugs as well. New β-lactamase inhibitor combination drugs in development have the potential to overcome some, but not all, of resistance from the most potent β-lactamases such as those found in CRE.

Extended-spectrum cephalosporins have been a cornerstone for treatment of serious Gram-negative infections for the past 20 years. (Drug-)Resistant Gram-negative infections are spreading into communities. Resistance often leaves carbapenem as the only effective antibacterial agent.

Fluoroquinolones are broad-spectrum antibiotics that are often given orally, making them convenient to use in both inpatients and outpatients. However, with increased use in a patient population drug-resistant strains rapidly evolve, rendering the drug ineffective. Increased use is also associated with an increase in infections caused by resistant, hypervirulent strains of Clostridium difficile.

Aminoglycosides are often used in combination with f-lactam drugs for the treatment of infections caused by Gram-negative bacteria. Despite growing resistance concerns, these drugs continue to be an important therapeutic option as a last resort against serious infections. However, they are rarely, if ever, used alone by clinicians because of concerns with resistance and their prolonged side effects.

Tetracyclines are not a first-line treatment option for serious Gram-negative infections; however, with limited efficacy of other drug classes, they are considered an option for treating serious infections. Glycylcyclines (i.e., tigecycline) are often considered for treatment of multidrug-resistant Gram-negative infections. Tigecycline is a drug that does not distribute evenly in the body, so it is often used in combination with other drugs depending upon the site of infection. Although relatively uncommon, there have been reported incidences of strains that are resistant to tigecycline.

Polymyxins are an older class that fell out of favor because of toxicity concerns. Now they are often used as a “last resort” agent for treatment of multi-drug resistant Gram-negative infections. Because these are generic drugs, there are limited contemporary data on dosimetry and efficacy. Additionally, there is some, but limited data regarding the detection of highly resistant strains.

Given the rapid increase in the number of drug-resistant strains of bacteria, there is an immediate need for new and efficient methods for identifying and karyotyping both clinical and non-clinical isolates of bacteria, particularly, those belonging to the ESKAPE group (Enterococcus faecium, Staphylococcus aureus, Klebsiella pneumoniae, Acinetobacter baumannii, Pseudomonas aeruginosa and Enterobacter species). (Boucher et al., Clinical Infectious Diseases, 48:1-12, 2009). Rapid and accurate pathogen identification is also needed to allow physicians to react and respond appropriately to infections, including those that are potentially life threatening. Currently, pathogen identification requires culture on solid medium (agar-based plate), followed by diagnostic analysis that normally requires additional rounds of replication in culture or purification of a specific bacterial product. At best, microbe identification requires multiple days during which additional levels of biosafety containment may be required depending on the overall classification of the pathogen. Second-generation versions of this biological, growth-based assay speed the time to detection of both microbial identification as well as resistance testing by using radiometric (e.g., Becton Dickinson's BACTEC™) or colorimetric/fluorometric (e.g., Becton Dickinson's MGIT™ and Biomerieux's BACT ALERT®) devices to measure metabolic products produced by growing bacteria, rather than waiting for the bacterial population to reach a density sufficient to be seen by the naked eye. However, these assay systems frequently face contamination problems, thus increasing the need for reprocessing and resulting in unnecessary delays (Tortoli et al., J. Clin. Microbiol., 40:607-610, 2002).

More recent approaches to speed the biological detection of drug resistant bacteria have focused on using bacteriophage to probe the effect an anti-microbial has on an isolate (Schofield et al., Bacteriophage, 2(2): 105-283, 2012 and WO 08/124119). The phages are used to infect the bacteria, hijack the hosts' cellular biosynthetic machines to replicate, thereby serve as tools for identifying the presence of particular strains of bacteria in clinical specimen. A variety of methods may be employed in the detection of the phage. One method relies on the use of nucleic acid amplification (U.S. Patent Pub. No. 2014-0256664 and WO 12/158502). In this method, drug susceptibility of M. tuberculosis is screened by analyzing real-time PCR products of mycobacteriophage D29 DNA.

A related method relies on infecting a secondary culture with the phage-harboring bacteria and analyzing the growth properties of the secondary culture. This method is typically used in identifying drug resistant M. tuberculosis. Exemplary commercial kits based on this indirect detection method are sold by Biotec, Inc. (Suffolk, UK) under the mark FASTPLAQUE-RESPONSE™. (Mole et al., J Med Microbiol., 56(Pt 10): 1334-9, 2007; Albert et al., J Appl Microbiol., 103(4):892-9, 2007). The kits are also provided with mycobacteriophage D29, however, in contrast to the direct PCR analysis of the D29 DNA, this method attempts to minimize false positives by using a virucide to eliminate phages that did not infect the bacteria. After screening for infected mycobacteria, the phage-infected M. tuberculosis is combined with a fast-replicating M. smegmatis and the mixture is then plated onto agar dishes. The assay system is based on the principle that M. smegmatis is efficiently cross-infected by D29 and forms clear and visible plaques on M. smegmatis bacterial lawns, such that each plaque represents an M. tuberculosis cell that was initially infected by D29. Thus, the assay quantitatively measures D29 replication in small pool of M. tuberculosis. Although an accurate and rapid test, this assay is too complicated and unwieldy for use in resource-poor settings because the analysis of viral growth by plaque formation on agar plates must be performed in a laboratory by a trained technician. Furthermore, the number of secondary fast-growing bacteria that are employable for this assay are limited, the assay cannot be customized or modified to screen for a large number of target bacterial species.

Similarly, variations on the original luciferase reporter assay (LRA), e.g., using engineered mycobacteriophage TM4, are also limited with regard to sensitivity of detection. See, Piuri et al., PLoS One, 2009; 4(3):e4870, wherein fluorophages (fluoromycobacteriophages) were able to detect only 50% of M. tuberculosis cells 16 h post-infection. Also, because this assay involves detection of fluorescent or luminescent markers expressed in small samples, the assays are limited with respect to types of samples that may be analyzed.

In summary, current approaches to identify drug resistant bacteria fail to satisfy today's need for efficient and practical means for phenotypic analysis of a large variety of bacteria, including, mixtures thereof, for e.g., on the basis of the type of resistance they harbor. There is therefore a pressing need for assay systems that are useful for screening susceptibility of particular strains of bacteria to antibacterial agents. Such assay technology could be effectively combined with the diagnosis, treatment and management of many human and veterinary diseases, such as, urinary tract infections, respiratory infections, bloodstream infections, etc. Such systems and assays could also be used in the screening of probiotics that can be used to supplement the growth of industrially-useful microbes, e.g., E. coli, R eutropha, S. carnosus, etc.

### BRIEF SUMMARY

It is therefore an object to provide less costly, more efficient, more specific, faster, more accessible, and better adaptable processes and apparatuses for selective microbe (e.g., bacterial) detection than provided by currently available technology. Accordingly, a method for determining bacterial resistance to antibiotics and a method for microbial species identification are provided. The methods exploit the intrinsic specificity of bacteriophages to their corresponding host bacteria.

In accordance with the foregoing, embodiments provide recombinant bacteriophages, a method for constructing and producing such recombinant bacteriophages, and methods for use of such recombinant bacteriophages for detecting target bacteria and/or determining drugs or antibiotics to which the target bacteria is/are resistant. The compositions and methods may also be adapted to screen for new pro-biotic agents that are useful for biosynthesis of enzymes, hormones, antibodies, nucleic acids, sugars, and other biomolecules at the laboratory level or on an industrial scale.

In accordance with an embodiment, products, kits, and methods that are capable of detecting specific types of bacteria, for example, by probing for the presence of specific molecule, e.g., a marker such as protein, in a targeted viable bacterium. Once the drug resistant strains are identified, the methods may, for example, be coupled with other techniques for identifying the molecular basis for drug resistance mechanism, e.g., genetic mutation, gene duplication, transformation, antibiotic degradation, etc. The present utilization of recombinant phages comprising genes of heterologous hydrolytic enzymes or non-coding RNAs, which are detectable by fluorescence assays, achieves the aforementioned objectives.

In some embodiments, a method is provided for simultaneously identifying a bacterial species and determining a susceptibility of the bacterial species to a test antimicrobial agent, comprising (a) providing a transforming phage specific to the bacterial species, wherein the transforming phage is an engineered or recombinant phage having a gene for encoding a marker, the gene being heterologous to both humans and the bacterial species; (b) preparing a first culture by culturing the bacterial species in the absence of the test antimicrobial agent to generate a primary test antimicrobial-free culture and culturing the primary test antimicrobial-free culture in the presence of the transforming phage; (c) preparing a second culture by culturing the bacterial species in the presence of the test antimicrobial agent to generate a primary test antimicrobial-containing culture; culturing the primary test antimicrobial-containing culture in the presence of the transforming phage; and (d) analyzing the first and second cultures to determine a level or activity of the marker in each of the first and second cultures.

In some embodiments, a detection of the marker in the first culture provides a positive identification of the bacterial species as the bacterial species to which the transforming phage is specific.

In some embodiments, a reduction in the level or activity of the marker in the second culture compared to the level or activity of the marker in the first culture indicates that the bacterial species is susceptible to the test antimicrobial agent.

In some embodiments, a uniformity or an increase in the level or activity of the marker in the second culture compared to the level or activity of the marker in the first culture indicates that the bacterial species is not susceptible to or is resistant to the test antimicrobial agent.

In some embodiments, a method is provided for determining a probiotic effect of a test agent on bacterial species, comprising (a) providing a transforming phage specific to the bacterial species, wherein the transforming phage is an engineered or recombinant phage having a gene for encoding a marker, the gene being heterologous to both humans and the bacterial species; (b) preparing a first culture by culturing the bacterial species in the absence of the test agent to generate a primary test agent-free culture and culturing the primary test agent-free culture in the presence of the transforming phage; (c) preparing a second culture by culturing the bacterial species in the presence of the test agent to generate a primary test agent-containing culture; culturing the primary test agent-containing culture in the presence of the transforming phage; and (d) analyzing the first and second cultures to determine a level or activity of the marker in each of the first and second cultures, wherein an increase in the level or activity of the marker in the second culture compared to the level or activity of the marker in the first culture indicates that the test agent has a probiotic effect.

In some embodiments, a method is provided for screening a test agent for antibacterial activity against a target bacterial specimen, comprising (a) providing a transforming phage specific to the target bacterial specimen, wherein the transforming phage is an engineered or recombinant phage having a gene for encoding a marker, the gene being heterologous to both humans and the bacterial species; (b) preparing a first culture by culturing the target bacterial specimen in the absence of the test agent to generate a primary test agent-free culture and culturing the primary test agent-free culture in the presence of the transforming phage; (c) preparing a second culture by culturing the target bacterial specimen in the presence of the test agent to generate a primary test agent-containing culture; culturing the primary test agent-containing culture in the presence of the transforming phage; and (d) analyzing the first and second cultures to determine a level or activity of the marker in each of the first and second cultures, wherein a reduction in the level or activity of the marker in the second culture compared to the level or activity of the marker in the first culture indicates that the test agent has anti-bacterial activity.

In some embodiments, a method is provided for determining the presence or absence of an antibiotic agent in a food sample, comprising (a) culturing the food sample in a plurality of bacterial cultures, wherein a first culture comprises a bacterial species that is susceptible to the antibiotic and a second culture comprises the same bacterial species, but is resistant to the antibiotic, thereby generating a plurality of primary cultures; (b) providing a transforming phage specific to the bacterial species, wherein the transforming phage is an engineered or recombinant phage having a gene for encoding a marker, the gene being heterologous to both humans and the bacterial species; (c) culturing the primary cultures of (a) in the presence or absence of the transforming phage which is specific to the bacterial species and which comprises a marker, thereby generating a plurality of secondary cultures, wherein a first transformed secondary culture is derived from a culture comprising bacteria that are susceptible to the antibiotic agent and a second transformed secondary culture comprises transformed bacteria that are resistant to the antibiotic agent; and (c) analyzing the first and second transformed secondary cultures to determine a level or activity of the marker in each of the first and second transformed secondary cultures, wherein a reduction in the level or activity of the marker in the first transformed secondary culture compared to the level or activity of the marker in the second transformed secondary culture indicates that the food sample comprises the antibiotic agent.

In some embodiments, a method is provided for determining a minimal inhibitory concentration (MIC) of an antibacterial agent against a target bacterial specimen, comprising (a) providing a transforming phage specific to the bacterial specimen, wherein the transforming phage is an engineered or recombinant phage having a gene for encoding a marker, the gene being heterologous to both humans and the bacterial specimen; (b) preparing a control culture by culturing the target bacterial specimen in the absence of the antibacterial agent to generate a primary culture, and subsequently culturing the primary culture in the presence of the transforming phage; (c) preparing an experimental group by preparing a plurality of experimental cultures comprising the target bacterial specimen and the antibacterial agent, wherein the antibacterial agent has a concentration that varies among the experimental cultures, and subsequently culturing the experimental cultures in the presence of the transforming phage; (d) analyzing cultures from the experimental group and the control culture to determine a level or activity of the marker in the cultures of the experimental group and the control culture, wherein the minimal concentration at which the antibacterial agent is capable of reducing the level or activity of the marker compared to a threshold level or activity of the marker in the control culture is indicative of the minimal inhibitory concentration of the agent against the target bacterial specimen.

In some embodiments, a method is provided for the diagnosis and treatment of a bacterial disease in a subject in need thereof, comprising (a) providing a subject sample comprising a bacterial species; (b) culturing the subject sample to generate a plurality of primary bacterial cultures; (c) providing a plurality of transforming phages, each of which is specific to a different bacterial species, wherein each transforming phage is an engineered or recombinant phage having a gene for encoding a unique marker, the gene being heterologous to both humans and the bacterial species; (d) culturing the primary bacterial cultures of (a) in the presence of a transforming phage of (c) to provide a plurality of secondary bacterial cultures, wherein the transforming phage varies among the secondary bacterial cultures; (e) analyzing the secondary bacterial cultures to determine the presence or absence of the unique marker in the secondary bacterial cultures; (f) correlating the detection of the unique marker with a presence of a bacterial species; (g) correlating the presence of the bacterial species with the bacterial disease; and (h) optionally administering, into the subject, an antibiotic agent that is specific to the detected bacterial species, thereby treating the bacterial disease.

In some embodiments of the above methods, the bacterial species or target bacterial specimen is selected from the group consisting of gram positive and Gram-negative bacteria.

In some embodiments of the above methods, the bacteria is selected from the group consisting of Acinetobacter baumannii, Bacillus anthracis, Bacillus cereus, Bordetella pertussis, Borrelia burgdorferi, Brucella aborus, Brucella canis, Brucella melitensis, Brucella suis, Campylobacter jejuni, Chlamydia pneumoniae, Chlamydia psittaci, Chlamydia trachomatis, Clostridium botulinum, Clostridium diicile, Clostridium perfringens, Clostridium tetani, Corynebacterium diphtheriae, Enterobacter sp., Enterococcus faecalis, vancomycin-resistant Enterococcus faecalis, Enterococcus faecium, Escherichia coli, enterotoxigenic Escherichia coli (ETEC), enteropathogenic Escherichia coli, E. coli O157:H7, Francisella tularensis, Haemophilus influenzae, Helicobacter pylori, Klebsiella pneumoniae, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Proteus, Pseudomonas aeruginosa, Rickettsia rickettsii, Salmonella typhi, Salmonella typhimurium, Shigella sonnei, Staphylococcus aureus, Staphylococcus epidermis, Staphylococcus saprophyticus, methicillin-resistant Staphylococcus aureus (MRSA), vancomycin-resistant Staphylococcus aureus (VSA), Streptococcus agalactiae, Streptococcus pneumoniae, Streptococcus pyogenes, Treponema pallidum, Vibrio cholerae, and Yersinia pestis or a combination thereof.

In some embodiments of the above methods, the bacteria are selected from those listed in Tables 1-3. In some embodiments of the above methods, the bacteria are selected from the group consisting of Bacillus anthracis, Bacillus subtilis, Bacillus thuringiensis, Escherichia coli, Lactobacillus delbrueckii, Lactobacillus plantarum, Lactococcus lactis, Listeria monocytogenes, Pseudomonas aeruginosa, Pseudomonas syringae, Klebsiella, Salmonella, Shigella, and Staphylococcus aureus.

In some embodiments of the above methods, the phage is a lytic or productive phage; a temperate or lysogenic phage; or a filamentous phage.

In some embodiments of the above methods, the phage is a lytic or productive phage selected from the group consisting of T4, T7, T3, and MS2.

In some embodiments of the above methods, the temperate or lysogenic phage is a, phage.

In some embodiments of the above methods, the phage is a filamentous phage selected from the group consisting of fl, fd, and M13.

In some embodiments of the above methods, the method may be practiced using a combination of various phages.

In some embodiments of the above methods, the gene is a fungal or plant-based gene.

In some embodiments of the above methods, the marker is a hydrolytic enzyme. In some embodiments, the hydrolytic enzyme is selected from the group consisting of cellulases, cutinases, esterases, lipases, phosphoesterases, restriction endonucleases, and proteases. In some embodiments, the hydrolytic enzyme is selected from the group consisting of collagenase, hyaluronidase, trypsin, chymotrypsin, pronase, elastase, DNase I, dispase, plasmin, bromelin, clostripain, thermolysin, neuraminidase, phospholipase, cholesterol esterase, subtilisin, papain, chymopapain, plasminogen activator, streptokinase, urokinase, fibrinolysin, serratiopeptidase, pancreatin, amylase, lysozyme, cathepsin-G, alkaline and acid phosphatases, esterases, decarboxylases, phospholipase D, P-xylosidase, β-D-fucosidase, thioglucosidase, β-D-galactosidase, α-D-galactosidase, α-D-glucosidase, β-D-glucosidase, β-D-glucuronidase, α-D-mannosidase, β-D-mannosidase, β-D-fructofuranosidase, β-D-glucosiduronase, and PMN leukocyte serine proteases.

In some embodiments, analyzing a culture to determine a level or activity of the hydrolytic enzyme comprises detecting hydrolysis of a substrate specific to the hydrolytic enzyme. In some embodiments, the substrate is an intramolecularly quenched fluorophore or an intramolecularly quenched chromogen. In some embodiments, the intramolecularly quenched fluorophore or the intramolecularly quenched chromogen is a compound having the formula:

Q-S-F
wherein,
S comprises a substrate specific to the hydrolytic enzyme;
F is a fluorophore, a chemiluminescent molecule, or a chromogen; and
Q is a quencher;
wherein S forms a link between Q and F, and hydrolysis of S severs the link, thereby reducing a quenching effect of Q upon F.

In some embodiments, F is a chromogen and detecting hydrolysis of the substrate comprises detecting a visual color change or acquiring a colorimetric or spectroscopic reading. In some embodiments, F is a fluorophore and detecting hydrolysis of the substrate comprises detecting fluorescence or acquiring a fluorometric reading. In some embodiments, F is selected from the group consisting of Alexa 405, FAM, Cy3, and Cy 5 and Q is selected from the group consisting of DABCYL, BHQ-1, BHQ-2, and BHQ-3. In some embodiments, F is a chemiluminescent molecule, and detecting hydrolysis of the substrate comprises performing a chemiluminescent assay.

In some embodiments, the substrate comprises a nucleic acid sequence and the hydrolytic enzyme is a restriction endonuclease specific to the nucleic acid sequence. In some embodiments, the substrate comprises a polypeptide, the hydrolytic enzyme is a protease, and the polypeptide is specific to the protease.

In some embodiments, the marker is a non-coding ribonucleic acid (RNA) heterologous to both humans and the bacterial species. In some embodiments, the non-coding RNA is self-splicing. In some embodiments, the non-coding RNA comprises two or more spliced introns.

In some embodiments, analyzing a culture to determine a level or activity of the non-coding RNA comprises amplifying the non-coding RNA. In some embodiments, the non-coding RNA is amplified by Reverse Transcriptase Helicase Dependent Amplification (RT-HDA) whereby a complementary deoxyribonucleic acid (cDNA) amplicon corresponding to the non-coding RNA is generated. In some embodiments, the non-coding RNA is amplified at least about 10,000 times, at least about 100,000 times, at least about 1×10⁶ times, at least about 1×10⁷ times, at least about 1×10⁸ times, at least about 1×10⁹ times, or at least about 1×10⁰ times.

In some embodiments, analyzing a culture to determine a level or activity of the non-coding RNA further comprises detecting the cDNA amplicon via an intramolecularly quenched probe which specifically binds the cDNA amplicon. In some embodiments, the intramolecularly quenched probe binds the cDNA amplicon at a region overlapping a site corresponding to a splice junction of the non-coding RNA. In some embodiments, the intramolecularly quenched probe comprises a fluorophore linked to a quencher via a nucleic acid linker of about 15 to about 30 bases in length. In some embodiments, detecting the cDNA amplicon further comprises disconnecting the fluorophore from the quencher by contacting a ribonuclease (RNase) to the intramolecularly quenched probe. In some embodiments, the RNase is RNase HII. In some embodiments, analyzing a culture to determine a level or activity of the non-coding RNA or the cDNA amplicon further comprises detecting fluorescence or acquiring a fluorometric reading of the fluorophore.

In some embodiments of the above methods, the methods may further comprise validating the detection results by detecting a secondary marker which is a nucleic acid selected from the group consisting of DNA, RNA or a combination thereof. In such embodiments wherein the initial detection is validated, the secondary nucleic acid marker may be detected with gel-electrophoresis, a nucleic acid amplification technique, such as polymerase chain reaction (PCR), quantitative polymerase chain reaction (qPCR) or a combination thereof.

### BRIEF DESCRIPTION OF THE DRAWINGS

The details of one or more embodiments of the invention are set forth in the accompanying drawings/tables and the description below. Other features, objects, and advantages of the invention will be apparent from the drawings/tables and detailed description, and from the claims.

FIG. 1 shows an exemplary illustration of an intramolecularly quenched substrate as described herein.

### DETAILED DESCRIPTION

Embodiments described herein provide methods and assays for diagnosis or detection of bacterial infectious agents and diseases using recombinant bacteriophages. The methods are suitable for the detection of bacterial infectious agents and also for determining drug resistance of such infectious agents. In addition, the methods are used to provide information concerning the susceptibility of the infectious agents to antimicrobial agents.

### A. Infectious Bacteria

Essentially any bacteria can be detected and the methods and compositions can be used for determining antibiotic susceptibility of bacteria or for screening a candidate antibiotic agent that exerts a desirable (e.g., antimicrobial or cytotoxic) effect on target bacteria.

In one embodiment, the bacteria are Gram-negative bacteria. Typical Gram-negative bacteria include proteobacteria such as E. coli, Salmonella, Pseudomonas, and Helicobacter, and cyanobacteria. When classified in connection with medicine, they include Pseudomonas aeruginosa and Hemophilus influenzae causing the disturbance of the respiratory system, Escherichia coli and Proteus mirabilis causing the disturbance of the urinary system, and Helicobacter pylori and Bacillus Gaertner causing the disturbance of the alimentary system and micrococci such as Neisseria meningitidis, Moraxella catarrhalis (causing disturbeance of, e.g., the central nervous system), and Neisseria gonorrhea (causing disturbance of the reproductive system).

In another embodiment, the bacteria are Gram-positive bacteria. By “Gram-positive bacteria” is meant a bacterium or bacteria that contain(s) teichoic acid (e.g., lipoteichoic acid and/or wall teichoic acid), or a functionally equivalent glycopolymer (e.g., a rhamnopolysaccharide, teichuronic acid, arabinogalactan, lipomannan, and lipoarabinomannan) in its cell wall. Non-limiting examples of functionally equivalent glycopolymers are described in Weidenmaier et al., Nature, 6:276-287, 2008. Additional examples of functionally equivalent glycopolymers are known in the art. In some embodiments, a Gram-positive bacterium is identified using the Gram staining method (e.g., generally including the steps of staining with crystal violet, treating with an iodine solution, decolorizing with alcohol, and counterstaining with safranine, wherein a Gram-positive bacterium retains the violet stain). Non-limiting examples of Gram-positive bacteria are described herein. Additional examples of Gram-positive bacteria are known in the art. Exemplary methods for detecting or identifying Gram-positive bacteria are described herein. Additional methods for detecting or identifying Gram-positive bacteria are known in the art.

The target bacteria include pathogenic bacteria that infect mammalian hosts (e.g., bovine, murine, equine, primate, feline, canine, and human hosts). In one embodiment, the bacteria infect and/or cause diseases in a human host. Examples of such pathogenic bacteria include, e.g., members of a bacterial species such as Bacteroides, Clostridium, Streptococcus, Staphylococcus, Pseudomonas, Haemophilus, Legionella, Mycobacterium, Escherichia, Salmonella, Shigella, Vibrio, or Listeria. Some clinically relevant examples of pathogenic bacteria that cause disease in a human host include, but are not limited to, Bacillus anthracis, Bacillus cereus, Bordetella pertussis, Borrelia burgdorferi, Brucella aborus, Brucella canis, Brucella melitensis, Brucella suis, Campylobacter jejuni, Chlamydia pneumoniae, Chlamydia psittaci, Chlamydia trachomatis, Clostridium botulinum, Clostridium dificile, Clostridium perfringens, Clostridium tetani, Corynebacterium diphtheriae, Enterococcus faecalis, vancomycin-resistant Enterococcus faecalis, Enterococcus faecium, Enterobacter spp., Enterobacter aerogenes, Enterobacter amnigenus, Enterobacter agglomerans, Enterobacter arachidis, Enterobacter asburiae, Enterobacter cancerogenous, Enterobacter cloacae, Enterobacter cowanii, Enterobacter dissolvens, Enterobacter gergoviae, Enterobacter helveticus, Enterobacter hormaechei, Enterobacter intermedius, Enterobacter kobei, Enterobacter ludwigii, Enterobacter mori, Enterobacter nimipressuralis, Enterobacter oryzae, Enterobacter pulveris, Enterobacter pyrinus, Enterobacter radicincitans, Enterobacter taylorae, Enterobacter turicensis, Enterobacter sakazakii, Enterobacter soli, Escherichia coli, enterotoxigenic Escherichia coli (ETEC), enteropathogenic Escherichia coli, E. coli O157:H7, Francisella tularensis, Haemophilus influenzae, Helicobacter pylori, Klebsiella spp., Klebsiella granulomatis, Klebsiella oxytoca, Klebsiella pneumoniae, Klebsiella terrigena, Klebsiella variicola, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Proteus, Pseudomonas aeruginosa, Rickettsia rickettsii, Salmonella typhi, Salmonella typhimurium, Serratia spp., Serratia aquatilis, Serratia entomophila, Serratia ficaria, Serratia fonticola, Serratia glossinae, Serratia grimesii, Serratia liquefaciens, Serratia marcescens, Serratia myotis, Serratia nematodiphila, Serratia odorifera, Serratia plymuthica, Serratia proteamaculans, Serratia quinivorans, Serratia rubidaea, Serratia symbiotica, Serratia ureilytica, Serratia vespertilionis, Shigella flexneri, Shigella dysenteriae, Shigella boydii, Shigella sonnei, Staphylococcus aureus, Staphylococcus epidermis, Staphylococcus saprophyticus, methicillin-resistant Staphylococcus aureus (MRSA), vancomycin-resistant Staphylococcus aureus (VSA), Streptococcus agalactiae, Streptococcus pneumoniae, Streptococcus pyogenes, Treponema pallidum, Vibrio cholerae, and Yersinia pestis.

In another embodiment, the infectious bacteria are selected from the group consisting of Clostridium difficile, Carbapenem-Resistant Enterobacteriaceae (CR-Klebsiella spp; CR-E. coli), and Neisseria gonorrhoeae. In another embodiment, the infectious bacteria is selected from the group consisting of multidrug-resistant Acinetobacter, drug-resistant Campylobacter, extended spectrum β-Lactamase (ESBL)-producing enterobacteriaceae, vancomycin-resistant Enterococcus, multidrug-resistant Pseudomonas aeruginosa, drug-resistant non-typhoidal Salmonella, drug-resistant Salmonella enterica serovar Typhi, drug-resistant Shigella, methicillin-resistant Staphylococcus aureus (MRSA), drug-resistant Streptococcus pneumoniae, and drug-resistant Tuberculosis. In another embodiment, the infectious bacteria are selected from the group consisting of vancomycin-resistant Staphylococcus aureus, erythromycin-resistant Group A Streptococcus, clindamycin-Resistant Group B Streptococcus.

In certain embodiments, the infectious agents are natively found in host subjects. In another embodiment, the infectious agents are invasive species that are foreign to host subjects. Preferably, the hosts are mammals, e.g., a rodent, a human, a livestock animal, a companion animal, or a non-domesticated or wild animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In an exemplary embodiment, the subject is a human.

The methods may be used to analyze infectious agents contained in a variety of samples including, e.g., biological sample, research test samples, environmental samples (such as water samples, including water samples selected from natural bodies of water, ponds, community water reservoirs, recreational waters, swimming pools, whirlpools, hot tubs, spas, water parks, naturally occurring fresh waters, and marine surface waters) and industrial samples (such as fermenting inoculums (such as Lactobacteria), chemical reagents, culture media, cleaning solutions)

Preferably, the sample is a biological sample comprising bodily fluids, e.g., sputum, tears, saliva, sweat, mucus, serum, semen, urine, stool, vomit, and blood. The sample may include e.g., cerebral spinal fluid (CSF), blood plasma, blood serum, lymph, lung lavage fluid, pleural fluid, etc. In some embodiments, the sample may be obtained from the subject using any known device or method, e.g., swabs, urethral catheters, aspirators, hypodermic needles, thin needle biopsies, hollow needle biopsies, punch biopsies, metabolic cages, and syringes.

In some embodiments, the biological sample is processed for use in the methods described herein. As a non-limiting example, a sputum or airway surface fluid (ASF) is collected in an appropriate vessel, such as a sterile specimen vial. The sample is solubilized using, for example, acetonitrile to a final concentration of about 60%, trifluoroacetic acid to a final concentration of about 0.1%, or using N-acetyl cysteine.

In certain embodiments, the biological sample may be manipulated to culture the bacteria contained therein. The term “culture” means either the cultured cells, the culture supernatant, the mixture thereof, or a culture filtrate if a liquid medium is used; if a solid medium is used, the term “culture” means the mixture of the cells and the medium on which they have grown. For example, if a liquid medium is used, the marker may be recovered from the culture mixture by the following procedures. When the full growth of the bacteria is attained, the culture mixture is subjected to treatment with the antibiotic and/or the phage. Such downstream processes may be intervened by one or more washing and/or separation steps comprising centrifugation or filtration, so as to obtain a crude bacterial preparation that is free from contaminants. The markers may be detected or analyzed at the cellular level (e.g., in situ) or after subjecting the cultures to further processing. For example, wherein the marker is a protein or a DNA in the cytosol, they may be extracted by disrupting cells using a suitable method such as grinding or ultrasonic treatment. Cells may be directly subjected to an ultrasonic treatment in a culture medium so as to disrupt the cells and a crude enzyme solution may be obtained by removing any insoluble matter from the treated solution.

If cultivation is performed on a solid medium, the markers may be analyzed by first manipulating the culture using the following procedure: water is added to the solid medium containing the cultured cells, and any insoluble matter is removed from the mixture either immediately or after disrupting the cells by a suitable means such as ultrasonic treatment. A crude marker preparation may be isolated from the crude lysate by conventional purification techniques, such as organic solvent fractionation, ammonium sulfate fractionation, dialysis, isoelectric precipitation and column chromatography, which may be used either independently or in combination. The level or activity of the marker may be determined using conventional methods, e.g., enzymatic assays for enzyme-like markers, nucleic acid hybridization and/or nucleic acid amplification, etc.

Depending on the objective, the cell cultures may be analyzed using routine techniques. For example, the bacteria may be cultured to logarithmic phase (MSSA USA300 and MRSA USA300) and peak logarithmic phase may be detected using conventional techniques, e.g., spectrophotometry. Use of logarithmic phase bacteria may be preferable because they are more likely to be adherent due to higher expression of adhesins and their peptidoglycan layer is likely to be less cross-linked and thick compared to stationary-phase cells and the cells are more metabolically active allowing for faster response to damage. However, optimal conditions may vary from strain to strain. Since different strains are often encountered in a clinical setting, this information is important for assessing the utility of the diagnostic methodology. Although it is contemplated herein that there will be strain variability, it is anticipated that the bacteria will behave similarly enough to permit the use of a single protocol for testing all the strains. This expectation is based on the fact that bacterial families (e.g., staphylococci) are genetically quite similar to each other and thus have similar cell structures, which will be the main component in their responsiveness to the particular phage.

In some embodiments, the methods and compositions are useful for the determination of susceptibility of a microbe, e.g., bacteria. As used herein, the term “susceptibility” refers to the degree to which a bacterial cell is affected by an antibiotic. That is, the cell may not be affected at all, it may have its growth and proliferation slowed or halted without its being killed or it may be killed. Susceptibility also refers to the degree a population of a bacterial species or strain is affected by an antibiotic. In this case, certain highly susceptible cells of the population may be very sensitive and may be killed by very low concentrations of the antibiotic, other less sensitive cells may have their growth and proliferation slowed while others may not be affected at all.

In a related embodiment, the methods and compositions are useful for identifying resistance of a microbe, e.g., bacteria, to an antimicrobial agent or an antibiotic. The term “resistant towards an antibiotic” herein means that a particular bacterial strain, often a mutant strain, is not killed, or killed significantly more slowly compared to the corresponding wild-type strain from which the strain is derived. Resistance can also be reflected by altered growth properties of the mutated and wild-type strains. For example, a low concentration of the antibiotic in the culture medium will prevent or significantly decrease the growth of wild-type strains while the growth of the mutated strains is not affected. The phenotype of a resistant strain, e.g., altered growth, cell division, metabolism, biofilm production, virulence, etc. may be determined using routine techniques, for e.g., growing wild-type and mutant strains under identical conditions to assess a change in the parameter being measured. Sensitive strains may be used as reference standards in the assessment of resistance (positive control).

In one embodiment, the methods are carried out by culturing a bacterial sample in presence of and in the absence of an antibiotic. The culture medium or fermentation medium may be modified or adjusted to meet the demands of the respective strains. Descriptions of culture media for various microorganisms are present in the “Manual of Methods for General Bacteriology” of the American Society for Bacteriology (Washington D.C., USA, 1981). The terms culture medium and fermentation medium or medium are interchangeable.

In its simplest sense, the culture medium contains at least one carbon source (e.g., glucose) and at least one nitrogen source (e.g., nitrate), optionally together with a phosphorus source, e.g., phosphoric acid, potassium phosphate or other phosphate salts. Preferably, the cultured medium is buffered for bacterial growth. The culture medium may additionally comprise salts, e.g., chlorides or -sulfates of metals such as, for example, sodium, potassium, magnesium, calcium and iron, such as, for example, magnesium sulfate or iron sulfate, which promote growth and/or metabolic activity. Finally, essential growth factors such as amino acids, for example homoserine and vitamins, for example thiamine, biotin or pantothenic acid, may be added to the culture media, depending on necessity. See, U.S. Pat. No. 9,074,229.

A starter sample containing the bacteria be added to the culture in the form of a single batch or be fed in during the cultivation in a suitable manner, e.g., every 2-4 hours or every 1-3 hours, or every 1, 2, 3, or 4 hours.

The pH of the culture can be controlled by employing basic compounds such as sodium hydroxide, potassium hydroxide, ammonia or aqueous ammonia, or acidic compounds such as phosphoric acid or sulfuric acid in a suitable manner. The pH is generally adjusted to a value of from 6.0 to 8.5, preferably 6.5 to 8. To control foaming, it is possible to employ antifoams such as, for example, fatty acid polyglycol esters. To maintain the stability of bacteria, it is possible to add to the medium suitable selective substances such as, for example, inducers such as isopropyl β-D-1-thiogalactopyranoside (IPTG). The fermentation is preferably carried out under aerobic conditions. In order to maintain these conditions, oxygen or oxygen-containing gas mixtures such as, for example, air are introduced into the culture. In batch or fed-batch processes, the cultivation is preferably continued until an amount of the desired density of the microbes is reached. Detection is carried out spectrophotometrically (absorption, fluorescence). This aim is normally achieved within 2 hours to 160 hours. In continuous processes, longer cultivation times are possible. The activity of the microorganisms results in a concentration (accumulation) of the various markers in the fermentation medium and/or in the cells of the microbes.

Examples of suitable fermentation media can be found inter alia in the U.S. Pat. Nos. 5,770,409; 5,275,940; 5,827,698; 5,756,345; and WO 2007/012078 and WO 2009/043803.

### B. Antibiotics

The aforementioned culture media may be supplemented with or without an antibiotic. As used herein, the term “antibiotic” or “antimicrobial agent” refers to a substance that inhibits the growth of or destroys microorganisms. Preferably, the antibiotic is useful in curbing the virulence of an infectious agent and/or treating an infectious disease. Antibiotic also refers to semi-synthetic substances wherein a natural form produced by a microorganism, e.g., yeast or fungus is subsequently structurally modified.

In another embodiment, the culture media may be supplemented with or without a probiotic substance. As used herein, the term “probiotic” refers to a substance that promotes the growth or metabolic activity of microorganisms, e.g., a micronutrient, a growth inducer substance, or a toxin removing substance.

Preferably, the antibiotic is selected from the group consisting of β-lactams (including, β-lactamase inhibitors and cephalosporins), fluoroquinolones, aminoglycosides, tetracyclines and/or glycylcyclines and/or polymyxins. Any combination of antimicrobial agents may also be tested, e.g., at least one β-lactam and at least one fluoroquinolone; at least one aminoglycoside and one cephalosporin; at least one β-lactam and one β-lactamase inhibitor, optionally together with an aminoglycoside, etc.

As used herein, the term “β-lactam” refers to any antibiotic agent which contains a β-lactam ring in its molecular structure. Representative examples include natural and semi-synthetic penicillins and penicillin derivatives, clavulanic acid, carbapenems, cephalosporins, cephamycins and monobactams. These drugs are metabolized by enzymes broadly referred to as “β-lactamases.” β-lactamases are organized into four molecular classes (A, B, C and D). Class A enzymes preferentially hydrolyze penicillins; class B enzymes include metalloenzymes that have a broader substrate profile than the others; class C enzymes are responsible for the resistance of Gram-negative bacteria to a variety of antibiotics; and class D enzymes are serine hydrolases, which exhibit a unique substrate profile.

Generally, β-lactams are classified and grouped according to their core ring structures, where each group may be divided to different categories. The term “penam” is used to describe the core skeleton of a member of a penicillin antibiotic, e.g., β-lactams containing a thiazolidine rings. Penicillins may include narrow spectrum penicillins, such as benzathine penicillin, benzylpenicillin (penicillin G), phenoxymethylpenicillin (penicillin V), procaine penicillin and oxacillin. Narrow spectrum penicillinase-resistant penicillins, such as methicillin, dicloxacillin and flucloxacillin. The narrow spectrum beta-lactamase-resistant penicillins may include temocillin. The moderate spectrum penicillins include for example, amoxicillin and ampicillin. The broad spectrum penicillins include the co-amoxiclav (amoxicillin+clavulanic acid). Finally, the penicillin group also includes the extended spectrum penicillins, for example, azlocillin, carbenicillin, ticarcillin, mezlocillin and piperacillin. Synthetic penicillin derivative includes, for example, faropenem.

β-lactams containing pyrrolidine rings are named carbapenams. The carbapenems group includes: biapenem, doripenem, ertapenem, imipenem, meropenem, panipenem and PZ-601.

Cephalosporins and cephamycins include cephalexin, cephalothin, cefazolin, cefaclor, cefuroxime, cefamandole, cefotetan, cefoxitin, cefotaxime, and cefpodoxime. Fourth generation cephalosporins, which are active against Gram-positive bacteria, include the cefepime and cefpirome. The cephalosporin class may further include: cefadroxil, cefixime, cefprozil, cephalexin, cephalothin, cefuroxime, cefamandole, cefepime and cefpirome. Cephamycins include, for example, cefoxitin, cefotetan, cefmetazole and flomoxef.

An example of carbacephems is loracarbef. Monobactams, which are active against Gram-negative bacteria include, for example, tigemonam, nocardicin A and tabtoxin. Synthetic cephems include, for example, clavulanic acid and oxacephems such as moxalactam and flomoxef.

Fluoroquinolones act by inhibiting enzymes that are essential for bacterial DNA replication. Representative examples of includes, ciprofloxacin, garenoxacin, gatifloxacin, gemifloxacin, levofloxacin, and moxifloxacin.

Aminoglycosides possess bactericidal activity against most Gram-negative aerobic and facultative anaerobic bacilli. Representative examples include, for e.g., kanamycin, amikacin, tobramycin, dibekacin, gentamicin, sisomicin, netilmicin, neomycin B, neomycin C, neomycin E (paromomycin) and streptomycin, including, synthetic derivatives clarithromycin and azithromycin.

Tetracyclines are a subclass of polyketides having an octahydrotetracene-2-carboxamide skeleton. They may be naturally-occurring (e.g., tetracycline, chlortetracycline, oxytetracycline, demeclocycline) or semi-synthetic (e.g., lymecycline, meclocycline, methacycline, minocycline, rolitetracycline). Glycylcyclines (e.g., minocycline/tigecycline) are derived from tetracyclines.

Polymyxins are polypeptide antibiotics that are active against Gram-negative bacteria such as E. coli and P. aeruginosa. Only polymyxin B and polymyxin E (colistin) are used clinically.

In practicing the methods the media may be supplemented with one or more of the aforementioned antibiotics. The concentration of the antibiotic may vary depending upon the antibiotic and the type of strain tested. Preferably, the dose of the antibiotic is equal to or greater than the minimum inhibitory concentration (MIC) of the particular antibiotic on the particular strain. Methods for determining MICs are known in the art (see, Andrews et al., J Antimicrob Chemother., 48 Suppl 1:5-16, 2001). A representative chart of MICs for 40 or so antimicrobial agents on four bacterial species (E. coli, S. aureus, P. aeruginosa, and E. faecalis) is shown in Table 3 of the Report published by European Committee for Antimicrobial Susceptibility Testing (EUCAST) entitled “Determination of minimum inhibitory concentrations (MICs) of antibacterial agents by broth dilution” (European Society of Clinical Alicrohiologv and Infectious Diseases CMI, 9, 1-7, 2003).

Generally, the concentration of the antibiotic may be increased for identifying or detecting resistant strains, e.g., by at least 2-fold, at least 5-fold, at least 10-fold, at least 20-fold, at least 50-fold, at least 100-fold, at least 300-fold or even 1000-fold over the baseline MIC. This is particularly effective in instances where the target bacteria and the MIC of the antibiotic on the bacteria are already known. For instance, for E. coli, the MIC for most antibiotics may range from about 0.01 mg/L to about 10 mg/L; however, resistant strains may not be susceptible until the concentration is increased, e.g., 10-fold (i.e. 1 log fold)-1000 fold (i.e., 3-log fold) over the base-line levels. In this regard, the final antibiotic concentration may be adjusted accordingly.

Purely for illustrative purposes, the following dosages may be employed—for testing the resistance of bacteria to β-lactams such as amoxicillin, the concentration may range from about 2 mg/L to about 40 mg/L, particularly from about 5 mg/L to about 20 mg/L. See, U.S. Pat. No. 9,347,888. On the other hand, for testing the resistance of bacteria to cloxacillin, the concentration may range between about 25 mg/L and about 300 mg/L. For carbapenem, the concentration may range between 0.05 and 32 mg/L. This includes a range between about 2 mg/L to about 32 mg/L for faropenem and from about 0.05 mg/L to about 2 mg/L for doripenem (see, Woodman et al., J Med Microbiol., 19(1):15-23, 1983). For cephalosporins, the concentration may range between about 1 mg/L to about 20 mg/L, preferably from about 4 mg/L to about 16 mg/L (see, Waterworth, J Clin Pathol, 35:1177-1180, 1982).

More particularly, the antibiotics may be used in a concentration of any one of 0.1 μg/mL, 0.5 μg/mL, 1 μg/mL, 2 μg/mL, 3 μg/mL, 4 μg/mL, 5 μg/mL, 6 μg/mL, 7 μg/mL, 8 μg/mL, 9 μg/mL, 10 μg/mL, 11 μg/mL, 12 μg/mL, 13 μg/mL, 14 μg/mL, 15 μg/mL, 16 μg/mL, 17 μg/mL, 18 μg/mL, 19 μg/mL, 20 μg/mL, 21 μg/mL, 22 μg/mL, 23 μg/mL, 24 μg/mL, 25 μg/mL, 26 μg/mL, 27 μg/mL, 28 μg/mL, 29 μg/mL, 30 μg/mL, 31 μg/mL, 32 g/mL, 33 g/mL, 34 μg/mL, 35 μg/mL, 36 μg/mL, 37 μg/mL, 38 μg/mL, 39 μg/mL, 40 μg/mL, 41 μg/mL, 42 μg/mL, 43 μg/mL 44 μg/mL, 45 μg/mL, 50 μg/mL, 60 μg/mL, 70 μg/mL, 80 mg/m, 90 μg/mL, 100 μg/mL, 150 μg/mL, 200 μg/mL, 250 μg/mL, 300 μg/mL, 400 μg/mL, 500 μg/mL, or more. For example, imipenem and ertapenem may be used in the concentrations of 50, 30, 20, 15, 10, 5 and 1 μg/mL. The dosages may be adjusted similarly for combination of antibiotics, e.g., by first determining MICs (combined agents) for wild-type strains and gradually increase the dosages to identify resistant strain(s).

The bacteria are cultured in presence or absence of the antibiotic for specified time periods, e.g., between 0 and 2 hours, 2 hours to 160 hours, particularly between 8 hours to 24 hours, especially between 10 hours to 16 hours. The bacteria may be at their growth phase or stationary phase prior to contact with the bacteriophage. The growth phase is a period characterized by cell doubling, wherein the number of cells in the culture grows exponentially. The stationary phase results from both growth of new bacteria and death of senescent cells, often due to a growth-limiting factor such as the depletion of an essential nutrient or accumulation of waste. Preferably, the bacteria are in growth phase prior to inoculation with the bacteriophage. Methods for determining growth phases of bacteria are known in the art. See, Hall et al., Mol Biol Evol., 31(1):232-8, 2014.

In one embodiment, the bacteria are treated with the antibiotic prior to inoculating with the bacteriophage. The primary culture may be optionally washed, e.g., with a wash buffer, prior to inoculation. Depending on the density of the surviving culture, the primary culture or a wash pellet thereof (obtained after centrifugation of the primary culture) may be re-grown in fresh native media (or antibiotic containing media) that has been inoculated with the bacteriophage.

In another embodiment, the bacteria are inoculated with the bacteriophage simultaneously with treatment with the antibiotic agent. This embodiment may be particularly suited for non-lytic phages.

### C. Phases

Embodiments of the instant methods utilize host-specific bacteriophages. As used herein, the term “bacteriophage” has its conventional meaning as understood in the art, e.g., a virus that selectively infects one or more bacteria. Many bacteriophages are specific to a particular genus or species or strain of bacteria. The term “bacteriophage” is synonymous with the term “phage.” Bacteriophages may include, but are not limited to, bacteriophages that belong to any of the following virus families: Corticoviridae, Cystoviridae, Inoviridae, Leviviridae, Microviridae, Myoviridae, Podoviridae, Siphoviridae, or Tectiviridae. The bacteriophage may be a lytic bacteriophage or a temperate bacteriophage or a filamentous bacteriophage. A lytic bacteriophage is one that follows the lytic pathway through completion of the lytic cycle, rather than entering the lysogenic pathway. A lytic bacteriophage undergoes viral replication leading to lysis of the cell membrane, destruction of the cell, and release of progeny bacteriophage particles capable of infecting other cells. A temperate bacteriophage is one capable of entering the lysogenic pathway, in which the bacteriophage becomes a dormant, passive part of the cell's genome through prior to completion of its lytic cycle. A filamentous bacteriophage contains a circular single-stranded deoxyribonucleic acid (ssDNA) genome packaged into long filaments. These phages do not reproduce by lysing bacteria; instead, they are secreted into the environment without killing the host.

In one embodiment, the phage is a lytic or productive phage (e.g., T4, T7, T3, and MS2). In another embodiment, the phage is a temperate or lysogenic phage (e.g., λ phage). In yet another embodiment, the phage is a filamentous phage (e.g., fl, fd, and M13). A combination of various phages may also be employed. Phage display techniques are known in the art, e.g., U.S. Pat. Nos. 8,685,893; 7,811,973; and U.S. Patent Publication No. 2002-0044922. Preferably, the phages are capable of transforming the host bacteria. As used herein, the term “transformation” means an introduction of DNA into a host cell such that DNA can be replicated as an extra-chromosomal element or by chromosomal integration. That is, transformation refers to synthetic alteration of genes by introducing a foreign DNA into the cell. As is recognized in the art, the DNA of most bacteria is contained in a single circular molecule, called the bacterial chromosome and one or more plasmids.

The phage is an engineered or a recombinant bacteriophage that serves as a vector for a gene that is foreign to the native phage. As used herein, the term “recombinant phage” or “engineered phage” is one that contains a nucleic acid sequence that is not naturally occurring or has a sequence that is made by an artificial combination of two otherwise separated segments of sequence. This artificial combination may be accomplished by chemical synthesis or artificial manipulation of isolated segments of nucleic acids, for example, by genetic engineering techniques or the use DNA transposition. Similarly, a recombinant protein is one encoded by a recombinant nucleic acid molecule. The term recombinant bacteriophage includes bacteriophages that have been altered solely by insertion of a nucleic acid, such as by inserting a nucleic acid encoding a heterologous protein that serves as a reporter or indicator molecule.

In certain embodiments, the phages are purified phages. The term purified does not require absolute purity; rather, it is intended as a relative term. A purified molecule is one in which the molecule is more enriched than it is in its natural environment, such as a preparation in which the molecule represents at least 50%, at least 60%, at least 80%, at least 90%, at least 99% or greater content of the total content of similar molecules within the sample. For example, a purified sample of recombinant phage is one in which the recombinant phage represents at least 50% of all bacteriophages within the sample.

A listing of pathogenic bacterial genera and their known host-specific bacteriophages is presented in the following paragraphs and preferred types of bacteria-phage pairs are provided in Tables 1-3. Synonyms and spelling variants are indicated in parentheses. Homonyms are repeated as often as they occur (e.g., D, D, d). Unnamed phages are indicated by “NN” beside their genus.

Bacteria of the genus Actinomyces are infected by the following phage: Av-1, Av-2, Av-3, BF307, CT1, CT2, CT3, CT4, CT6, CT7, CT8 and 1281.

Bacteria of the genus Aeromonas are infected by the following phage: AA-1, Aeh2, N, PM1, TP446, 3, 4, 11, 13, 29, 31, 32, 37, 43, 43-10T, 51, 54, 55R 1, 56, 56RR2, 57, 58, 59.1, 60, 63, Aeh1, F, PM2, 1, 25, 31, 40RR2.8t, (syn=44R), (syn=44RR2.8t), 65, PM3, PM4, PM5 and PM6.

Bacteria of the genus Bacillus are infected by the following phage: A, aiz1, Al-K-I, B, BCJA1, BC1, BC2, BLL1, BL1, BP142, BSL1, BSL2, BS1, BS3, BS8, BS15, BS18, BS22, BS26, BS28, BS31, BS104, BS105, BS106, BTB, B1715V1, C, CK-1, Col1, Cor1, CP-53, CS-1, CS1, D, D, D, D5, ent1, FP8, FP9, PSI, FS2, FS3, FS5, FS8, FS9, G, GH8, GT8, GV-1, GV-2, GT-4, g3, g12, g13, g14, g16, g17, g21, g23, g24, g29, H2, ken1, KK-88, Kum1, Kyu1, J7W-1, LP52, (syn=LP-52), L7, Mex1, MJ-1, mor2, MP-7, MP10, MP12, MP14, MP15, Neo1, No 2, N5, N6P, PBC1, PBLA, PBP1, P2, S-a, SF2, SF6, Sha1, Sil1, SPO2, (syn=ΦSPP1), SPβ, STI, ST1, SU-11, t, Tb1, Tb2, Tb5, Tb10, Tb26, Tb51, Tb53, Tb55, Tb77, Tb97, Tb99, Tb560, Tb595, Td8, Td6, Td15, Tg1, Tg4, Tg6, Tg7, Tg9, Tg10, Tg11, Tg13, Tg15, Tg21, Tin1, Tin7, Tin8, Tin13, Tm3, Toc1, Tog1, tol1, TP-1, TP-10vir, TP-15c, TP-16c, TP-17c, TP-19, TP35, TP51, TP-84, Tt4, Tt6, type A, type B, type C, type D, type E, Tφ3, VA-9, W, wx23, wx26, Yun1, α, γ, ρ11, φmed-2, φT, φμ-4, φ3T, φ75, φ105, (syn=φ105), 1A, 1B, 1-97A, 1-97B, 2, 2, 3, 3, 3, 5, 12, 14, 20, 30, 35, 36, 37, 38, 41C, 51, 63, 64, 138D, I, II, IV, NN-Bacillus (13), ale1, AR1, AR2, AR3, AR7, AR9, Bace-11, (syn=11), Bastille, BL1, BL2, BL3, BL4, BL5, BL6, BL8, BL9, BP124, BS28, BS80, Ch, CP-51, CP-54, D-5, dar1, den1, DP-7, ent2, FoS1, FoS2, FS4, FS6, FS7, G, gal1, gamma, GE1, GF-2, GS1, GT-1, GT-2, GT-3, GT-4, GT-5, GT-6, GT-7, GV-6, g15, I9, I10, IS1, K, MP9, MP13, MP21, MP23, MP24, MP28, MP29, MP30, MP32, MP34, MP36, MP37, MP39, MP40, MP41, MP43, MP44, MP45, MP47, MP50, NLP-1, No. 1, N17, N19, PBS1, PK1, PMB1, PMB12, PMJ1, S, SPO1, SP3, SP5, SP6, SP7, SP8, SP9, SP10, SP-15, SP50, (syn=SP-50), SP82, SST, sub1, SW, Tg8, Tg12, Tg13, Tg14, thu1, thu4, thu5, Tin4, Tin23, TP-13, TP33, TP50, TSP-1, type V, type VI, V, Vx, β22, φe, φNR2, φ25, φ63, 1, 1, 2, 2C, 3NT, 4, 5, 6, 7, 8, 9, 10, 12, 12, 17, 18, 19, 21, 138, III, 4 (B. megaterium), 4 (B. sphaericus), AR13, BPP-10, BS32, BS107, B1, B2, GA-I, GP-10, GV-3, GV-5, g8, MP20, MP27, MP49, Nf, PP5, PP6, SF5, Tg18, TP-1, Versailles, φ15, φ29, 1-97, 837/IV, NN-Bacillus (1), Bat10, BSL10, BSL11, BS6, BS11, BS16, BS23, BS101, BS102, g18, mor1, PBL1, SN45, thu2, thu3, Tm1, Tm2, TP-20, TP21, TP52, type F, type G, type IV, NN-Bacillus (3), BLE, (syn=θc), BS2, BS4, BS5, BS7, B10, B12, BS20, BS21, F, MJ-4, PBA12, AP50, AP50-04, AP50-11, AP50-23, AP50-26, AP50-27 and Bam35. The following Bacillus-specific phage are defective: DLP10716, DLP-1946, DPB5, DPB12, DPB21, DPB22, DPB23, GA-2, M, No. 1M, PBLB, PBSH, PBSV, PBSW, PBSX, PBSY, PBSZ, phi, SPα, type 1 and μ.

Bacteria of the genus Bacteroides are infected by the following phage: ad12, Baf-44, Baf-48B, Baf-64, Bf-1, Bf-52, B40-8, F1, β1, φAl, φBr01, φBr02, 11, 67.1, 67.3, 68.1, NN-Bacteroides (3), Bf42, Bf71, and BF-41.

Bacteria of the genus Bordetella are infected by the following phage: 134 and NN-Bordetella (3).

Bacteria of the genus Borrellia are infected by the following phage: NN-Borrelia (1) and NN-Borrelia.

Bacteria of the genus Brucella are infected by the following phage: A422, Bk, (syn=Berkeley), BM29, FO1, (syn=FO1), (syn=FQ1), D, FP2, (syn=FP2), (syn=FD2), Fz, (syn=Fz75/13), (syn=Firenze 75/13), (syn=Fi), F1, (syn=F1), F1m, (syn=F1m), (syn=Fim), F1U, (syn=F1U), (syn=FiU), F2, (syn=F2), F3, (syn=F3), F4, (syn=F4), F5, (syn=F5), F6, F7, (syn=F7), F25, (syn=F25), (syn=f25), F25U, (syn=F25u), (syn=F25U), (syn=F25V), F44, (syn=F44), F45, (syn=F45), F48, (syn=F48), I, Im, M, MC/75, M51, (syn=M85), P, (syn=D), S708, R, Tb, (syn=TB), (syn=Tbilisi), W, (syn=Wb), (syn=Weybridge), X, 3, 6, 7, 10/1, (syn=10), (syn=F8), (syn=F8), 12m, 24/II, (syn=24), (syn=F9), (syn=F9), 45/III, (syn=45), 75, 84, 212/XV, (syn=212), (syn=F10), (syn=F10), 371/XXIX, (syn=371), (syn=F11), (syn=P 1) and 513.

Bacteria of the genus Burkholderia are infected by the following phage: CP75.

Bacteria of the genus Campylobacter are infected by the following phage: C type, NTCC12669, NTCC12670, NTCC12671, NTCC12672, NTCC12673, NTCC12674, NTCC12675, NTCC12676, NTCC12677, NTCC12678, NTCC12679, NTCC12680, NTCC12681, NTCC12682, NTCC12683, NTCC12684, 32f, 111c, 191, Vfi-6, (syn=V19), Vfv-3, V2, V3, V8, V16, (syn=Vfi-1), V19, V20(V45), V45, (syn=V-45) and NN-Campylobacter (1).

Bacteria of the genus Chlamydia are infected by the following phage: Chp1.

Bacteria of the genus Clostridium are infected by the following phage: CAK1, CA5, Ca7, CEβ, (syn=1C), CEγ, Cld1, c-n71, c-203 Tox−, DEβ, (syn=1D), (syn=1Dtox+), HM3, KM1, KT, Ms, NA1, (syn=Naltox+), PA1350e, Pfô, PL73, PL78, PL81, P1, P50, P5771, P19402, 1Ctox+, 2Ctox−, 2D, (syn=2Dtox+), 3C, (syn=3Ctox+), 4C, (syn=4tox+), 56, III-1, NN-Clostridium (61), NB1tox-α1, CA1, HMT, HM2, PF1, P-23, P-46, Q-05, Q-06, Q-16, Q-21, Q-26, Q-40, Q-46, S111, SA02, WA01, WA03, W111, W523, 80, C, CA2, CA3, CPT1, CPT4, cl, c4, c5, HM7, H11/A1, H18/A1, H22/S23, H158/A1, K2/A, K21/S23, ML, NA2tox-, Pf2, P3, Pf4, S9/S3, S41/A1, S44/S23, α2, 41, 112/S23, 214/S23, 233/A1, 234/S23, 235/S23, II-1, II-2, II-3, NN-Clostridium (12), CA1, F1, K, S2, 1, 5 and NN-Clostridium (8).

Bacteria of the genus Corynebacterium are infected by the following phage: CGI1 (defective), A, A2, A3, A110, A128, A133, A137, A139, A155, A182, B, BF, B17, B18, B51, B271, B275, B276, B277, B279, B282, C, cap1, CC1, CG1, CG2, CG33, CL31, Cog, (syn=CG5), D, E, F, H, H-1, hq1, hq2, I1/H33, I1/H33, J, K, K, (syn=Ktox−), L, L, (syn=Ltox+), M, MC-1, MC-2, MC-3, MC-4, MLMa, N, O, ov1, ov2, ov3, P, P, P, RP6, RS29, S, T, U, UB1, ub2, UH1, UH3, uh3, uh5, uh6, β, (syn=βtox+), βlav64, βvir, γ, (syn=γtox−), γ19, δ, (syn=δtox+), ρ, (syn=ρtox−), φ9, φ984, ω, 1A, 1/1180, 2, 2/1180, 5/1180, 5ad/9717, 7/4465, 8/4465, 8ad/10269, 10/9253, 13/9253, 15/3148, 21/9253, 28, 29, 55, 2747, 2893, 4498 and 5848.

Bacteria of the genus Enterococcus are infected by the following phage: DF78, F1, F2, 1, 2, 4, 14, 41, 867, D1, SB24, 2BV, 182, 225, C2, C2F, E3, E62, DS96, H24, M35, P3, P9, SB101, S2, 2BII, 5, 182a, 705, 873, 881, 940, 1051, 1057, 21096C, NN-Enterococcus (1), PE1, PI, F3, F4, VD13, 1, 200, 235 and 341.

Bacteria of the genus Erysipelothrix are infected by the following phage: NN-Erysipelothrix (1).

Bacteria of the genus Escherichia are infected by the following phage: BW73, B278, D6, D108, E, E1, E24, E41, FI-2, FI-4, FI-5, HI8A, HI8B, i, MM, Mu, (syn=mu), (syn=Mu1), (syn=Mu-1), (syn=MU-1), (syn=MuI), (syn=mu), O25, PhI-5, Pk, PSP3, P1, PID, P2, P4 (defective), S1, Wφ, φK13, φR73 (defective), φ1, φ2, φ7, φ92, ψ (defective), 7A, 8φ, 9φ, 15 (defective), 18, 28-1, 186, 299, NN-Escherichia (2), AB48, CM, C4, C16, DD-VI, (syn=Dd-Vi), (syn=DDVI), (syn=DDVi), E4, E7, E28, F11, F13, H, H1, H3, H8, K3, M, N, ND-2, ND-3, ND4, ND-5, ND6, ND-7, Ox-1, (syn=OX1), (syn=11F), Ox-2, (syn=Ox2), (syn=OX2), Ox-3, Ox-4, Ox-S, (syn=OX5), Ox-6, (syn=66F), (syn=φ66t), (syn=φ66t−), O111, PhI-1, RB42, RB43, RB49, RB69, S, Sal-1, Sal-2, Sal-3, Sal-4, Sal-5, Sal-6, TC23, TC45, TuII*-6, (syn=TuII*), TuII*-24, TuII*46, TuII*-60, T2, (syn=gamma), (syn=γ), (syn=PC), (syn=P.C.), (syn=T-2), (syn=T2), (syn=P4), T4, (syn=T-4), (syn=T4), T6, T35, α1, 1, 1A, 3, (syn=Ac3), 3A, 3T+, (syn=3), (syn=M1), 5φ, (syn=φ5), 9266Q, CFO103, HK620, J, K, K1F, m59, no. A, no. E, no. 3, no. 9, N4, sd, (syn=Sd), (syn=SD), (syn=Sd), (syn=sd), (syn=SD), (syn=CD), T3, (syn=T-3), (syn=T3), T7, (syn=T-7), (syn=T7), WPK, W31, ΔH, φC3888, φK3, φK7, φK12, φV-1, Φ04-CF, Φ05, Φ06, Φ07, φ1, φ1.2, φ20, φ95, φ263, φ1092, φI, φII, (syn=φW), Ω8, 1, 3, 7, 8, 26, 27, 28-2, 29, 30, 31, 32, 38, 39, 42, 933W, NN-Escherichia (1), Esc-7-11, AC30, CVX-5, C1, DDUP, EC1, EC2, E21, E29, F1, F26S, F27S, Hi, HK022, HK97, (syn=ΦHK97), HK139, HK253, HK256, K7, ND-i, no. D, PA-2, q, S2, T1, (syn=α), (syn=P28), (syn=T-1), (syn=T1), T3C, T5, (syn=T-5), (syn=T5), UC-1, w, β4, γ2, λ, (syn=lambda), (syn=Φλ), ΦD326, φγ, Φ06, Φ7, Φ10, φ80, χ, (syn=χ1), (syn=φχ), (syn=φχ1), 2, 4, 4A, 6, 8A, 102, 150, 168, 174, 3000, AC6, AC7, AC28, AC43, AC50, AC57, AC81, AC95, HK243, K10, ZG/3A, 5, 5A, 21EL, H19-f and 933H.

Bacteria of the genus Fusobacterium are infected by the following phage: NN-Fusobacterium (2), fv83-554/3, fv88-531/2, 227, fv2377, fv2527 and fv8501.

Bacteria of the genus Haemophilus are infected by the following phage: HP1 (Haemophilus phage HP1), S2 and N3.

Bacteria of the genus Helicobacter are infected by the following phage: HP1 (Helicobacter pylori phage HP1) and NN-Helicobacter (1).

Bacteria of the genus Klebsiella are infected by the following phage: AIO-2, Kl4B, Kl6B, Kl9, (syn=Kl9), Kl14, Kl15, Kl21, Kl28, Kl29, Kl32, Kl33, Kl35, Kl106B, Kl171B, Kl181B, Kl832B, AIO-1, AO-1, AO-2, AO-3, FC3-10, K, Kl1, (syn=Kl1), Kl2, (syn=Kl2), Kl3, (syn=Kl3), (syn=Kl70/11), Kl4, (syn=Kl4), Kl5, (syn=Kl5), Kl6, (syn=Kl6), Kl7, (syn=Kl7), Kl8, (syn=Kl8), Kl19, (syn=Kl9), Kl27, (syn=Kl27), Kl31, (syn=Kl31), Kl35, Kl171B, II, VI, IX, CI-1, Kl4B, Kl8, Kl11, Kl12, Kl13, Kl16, Kl17, Kl18, Kl20, Kl22, Kl23, Kl24, Kl26, Kl30, Kl34, Kl106B, Kl165B, Kl328B, KLXI, K328, P5046, 11, 380, III, IV, VII, VIII, FC3-11, Kl2B, (syn=Kl2B), Kl25, (syn=Kl25), Kl42B, (syn=Kl42), (syn=Kl42B), Kl181B, (syn=Kl181), (syn=Kl181B), Kl765/1, (syn=Kl765/1), Kl842B, (syn=Kl832B), Kl937B, (syn=Kl937B), L1, φ28, 7, 231, 483, 490, 632 and 864/100.

Bacteria of the genus Leptospira are infected by the following phage: LE1, LE3, LE4 and NN-Leptospira (1).

Bacteria of the genus Listeria are infected by the following phage: A511, O1761, 4211, 4286, (syn=BO54), A005, A006, A020, A500, A502, A511, A118, A620, A640, B012, B021, B024, B025, B035, B051, B053, B054, B055, B056, B101, B110, B545, B604, B653, C707, D441, HSO47, H1OG, H8/73, H19, H21, H43, H46, H107, H108, H10, H163/84, H1312, H340, H387, H391/73, H684/74, H924A, PSA, U153, φMLUP5, (syn=P35), 00241, 00611, 02971A, 02971C, 5/476, 5/911, 5/939, 5/11302, 5/11605, 5/11704, 184, 575, 633, 699/694, 744, 900, 1090, 1317, 1444, 1652, 1806, 1807, 1921/959, 1921/11367, 1921/11500, 1921/11566, 1921/12460, 1921/12582, 1967, 2389, 2425, 2671, 2685, 3274, 3550, 3551, 3552, 4276, 4277, 4292, 4477, 5337, 5348/11363, 5348/11646, 5348/12430, 5348/12434, 10072, 11355C, 11711A, 12029, 12981, 13441, 90666, 90816, 93253, 907515, 910716 and NN-Listeria (15).

Bacteria of the genus Morganella are infected by the following phage: 47.

Bacteria of the genus Mycobacterium are infected by the following phage: I3, AG1, AL1, ATCC 11759, A2, B.C3, BG2, BK1, BK5, butyricum, B-1, B5, B7, B30, B35, Clark, C1, C2, DNAIII, DSP1, D4, D29, GS4E, (syn=GS4E), GS7, (syn=GS-7), (syn=GS7), IPα, lacticola, Legendre, Leo, L5, (syn=ΦL-5), MC-1, MC-3, MC-4, minetti, MTPH11, Mx4, MyF3P/59a, phlei, (syn=phlei 1), phlei 4, Polonus II, rabinovitschi, smegmatis, TM4, TM9, TM10, TM120, Y7, Y10, φ630, 1B, IF, 1H, 1/1, 67, 106, 1430, B1, (syn=Bol), B24, D, D29, F-K, F-S, HP, Polonus I, Roy, R1, (syn=R1-Myb), (syn=R1), 11, 31, 40, 50, 103a, 103b, 128, 3111-D, 3215-D and NN-Mycobacterium (1).

Bacteria of the genus Neisseria are infected by the following phage: Group I, group II and NP1.

Bacteria of the genus Nocardia are infected by the following phage: P8, NJ-L, NS-8, N5 and NN-Nocardia (1).

Bacteria of the genus Proteus are infected by the following phage: Pm5, 13vir, 2/44, 4/545, 6/1004, 13/807, 20/826, 57, 67b, 78, 107/69, 121, 9/0, 22/608, 30/680, Pm1, Pm3, Pm4, Pm6, Pm7, Pm9, Pm10, Pm11, Pv2, π1, φm, 7/549, 9B/2, 10A/31, 12/55, 14, 15, 16/789, 17/971, 19A/653, 23/532, 25/909, 26/219, 27/953, 32A/909, 33/971, 34/13, 65, 5006M, 7480b, VI, 13/3a, Clichy 12, π2600, φχ7, 1/1004, 5/742, 9, 12, 14, 22, 24/860, 2600/D52, Pm8 and 24/2514.

Bacteria of the genus Providencia are infected by the following phage: PL25, PL26, PL37, 9211/9295, 9213/9211b, 9248, 7/R49, 74761322, 7478/325, 7479, 7480, 9000/9402 and 9213/9211a.

Bacteria of the genus Pseudomonas are infected by the following phage: Pf1, (syn=Pf-1), Pf2, Pβ, PP7, PRR1, 7s, NN-Pseudomonas (1), AI-1, M-2, B17, B89, CB3, Col 2, Col 11, Col 18, Col 21, C154, C163, C167, C2121, E79, F8, ga, gb, H22, K1, M4, N2, Nu, PB-1, (syn=PB 1), pf16, PMN17, PP1, PP8, Psal, PsPI, PsP2, PsP3, PsP4, PsP5, PS3, PS17, PTB80, PX4, PX7, PYO1, PYO2, PYO5, PYO6, PYO9, PYO10, PYO13, PYO14, PYO16, PYO18, PYO19, PYO20, PYO29, PYO32, PYO33, PYO35, PYO36, PYO37, PYO38, PYO39, PYO41, PYO42, PYO45, PYO47, PYO48, PYO64, PYO69, PYO103, P1K, SLP1, SL2, S2, UNL-1, wy, Ya1, Ya4, Ya11, φBE, φCTX, φC17, φKZ, (syn=ΦKZ), φ-LT, Φmu78, φNZ, φPLS-1, φST-1, φW-14, φ-2, 1/72, 2/79, 3, 3/DO, 4/237, 5/406, 6C, 6/6660, 7, 7v, 7/184, 8/280, 9/95, 10/502, 11/DE, 12/100, 12S, 16, 21, 24, 25F, 27, 31, 44, 68, 71, 95, 109, 188, 337, 352, 1214, NN-Pseudomonas (23), A856, B26, CI-1, CI-2, C5, D, gh-1, F116, HF, H90, K5, K6, K104, K109, K166, K267, N4, N5, 06N-25P, PE69, Pf, PPN25, PPN35, PPN89, PPN91, PP2, PP3, PP4, PP6, PP7, PP8, PP56, PP87, PP 114, PP206, PP207, PP306, PP651, Psp231a, Pssy401, Pssy9220, ps1, PTB2, PTB20, PTB42, PX1, PX3, PX10, PX12, PX14, PYO70, PYO71, R, SH6, SH133, tf, Ya5, Ya7, φBS, ΦKf77, φ-MC, ΦmnF82, φPLS27, φPLS743, φS-1, 1, 2, 2, 3, 4, 5, 6, 7, 7, 8, 9, 10, 11, 12, 12B, 13, 14, 15, 14, 15, 16, 17, 18, 19, 20, 20, 21, 21, 22, 23, 23, 24, 25, 31, 53, 73, 119x, 145, 147, 170, 267, 284, 308, 525, NN-Pseudomonas (5), af, A7, B3, B33, B39, BI-1, C22, D3, D37, D40, D62, D3112, F7, F10, g, gd, ge, gf, Hw12, Jb19, KF1, L^{o}, OXN-32P, O6N-52P, PCH-1, PC13-1, PC35-1, PH2, PH51, PH93, PH132, PMW, PM13, PM57, PM61, PM62, PM63, PM69, PM105, PM 113, PM681, PM682, PO4, PP1, PP4, PP5, PP64, PP65, PP66, PP71, PP86, PP88, PP92, PP401, PP711, PP891, Pssy41, Pssy42, Pssy403, Pssy404, Pssy420, Pssy923, PS4, PS-10, Pz, SDI, SL1, SL3, SL5, SM, φC5, φC11, φC11-1, φC13, φC15, φMO, φX, φ04, φ11, φ240, 2, 2F, 5, 7m, 11, 13, 13/441, 14, 20, 24, 40, 45, 49, 61, 73, 148, 160, 198, 218, 222, 236, 242, 246, 249, 258, 269, 295, 297, 309, 318, 342, 350, 351, 357-1, 400-1, NN-Pseudomonas (6), G10, M6, M6a, L1, PB2, Pssy15, Pssy4210, Pssy4220, PYO12, PYO34, PYO49, PYO50, PYO51, PYO52, PYO53, PYO57, PYO59, PYO200, PX2, PX5, SL4, φ03, φ06 and 1214.

Bacteria of the genus Rickettsia are infected by the following phage: NN-Rickettsia (1).

Bacteria of the genus Salmonella are infected by the following phage: b, Beccles, CT, d, Dundee, f, Fels 2, GI, GIII, GVI, GVIII, k, K, i, j, L, O1, (syn=O-1), (syn=O1), (syn=O-I), (syn=7), O2, O3, P3, P9a, P10, Sab3, Sab5, San15, San17, SI, Taunton, ViI, (syn=Vi1), 9, NN-Salmonella (1), N-1, N-5, N-10, N-17, N-22, 11, 12, 16-19, 20.2, 36, 449C/C178, 966A/C259, a, B.A.O.R., e, G4, GIII, L, LP7, M, MG40, N-18, PSA68, P4, P9c, P22, (syn=P22), (syn=PLT22), (syn=PLT22), P22a1, P22-4, P22-7, P22-11, SNT-1, SNT-2, SP6, ViIII, ViIV, ViV, ViVI, ViVII, Worksop, ε15, ε34, 1, 37, 1(40), (syn=φ1[40]), 1, 422, 2, 2.5, 3b, 4, 5, 6, 14(18), 8, 14(6,7), 10, 27, 28B, 30, 31, 32, 33, 34, 36, 37, 39, 1412, SNT-3,7-11, 40.3, c, C236, C557, C625, C966N, g, GV, G5, G173, h, IRA, Jersey, M78, P22-1, P22-3, P22-12, Sab1, Sab2, Sab2, Sab4, San1, San2, San3, San4, San6, San7, San8, San9, San13, San14, San16, San18, San19, San20, San21, San22, San23, San24, San25, San26, SasL1, SasL2, SasL3, SasL4, SasL5, S1BL, SII, ViII, φ1, 1, 2, 3a, 3aI, 1010, NN-Salmonella (1), N-4, SasL6 and 27.

Bacteria of the genus Serratia are infected by the following phage: A2P, PS20, SMB3, SMP, SMP5, SM2, V40, V56, κ, DCP-3, (DCP-6, 3M, 10/1a, 20A, 34CC, 34H, 38T, 345G, 345P, 501B, SMB2, SMP2, BC, BT, CW2, CW3, CW4, CW5, L1232, L2232, L34, L.228, SLP, SMPA, V.43, σ, φCW1, ΦCP6-1, ΦCP6-2, ΦCP6-5, 3T, 5, 8, 9F, 10/1, 20E, 32/6, 34B, 34CT, 34P, 37, 41, 56, 56D, 56P, 60P, 61/6, 74/6, 76/4, 101/8900, 226, 227, 228, 229F, 286, 289, 290F, 512, 764a, 2847/10, 2847/10a, L.359 and SMB1,

Bacteria of the genus Shigella are infected by the following phage: Fsa, (syn=a), FSD2d, (syn=D2d), (syn=W2d), FSD2E, (syn=W2e), fv, F6, f7.8, H-Sh, PE5, P90, SflI, Sh, SHIII, SHIV, (syn=HIV), SHVI, (syn=HVI), SHVIII, (syn=HVIII), SKγ66, (syn=gamma 66), (syn=γ66), (syn=γ66b), SKIII, (syn=SIIIb), (syn=III), SKIV, (syn=SIVa), (syn=IV), SKIVa, (syn=SIVAn), (syn=IVA), SKVI, (syn=KVI), (syn=SVI), (syn=VI), SKVIII, (syn=SVIII), (syn=VIII), SKVIIIA, (syn=SVIIIA), (syn=VIIIA), STVI, STIX, STXI, STXII, S66, W2, (syn=D2c), (syn=D20), φI, φIV1, 3-SO-R, 8368-SO-R, F7, (syn=FS7), (syn=K29), F10, (syn=FS10), (syn=K31), I1, (syn=alfa), (syn=FSα), (syn=K18), (syn=α), I2, (syn=a), (syn=K19), SG35, (syn=G35), (syn=SO-35/G), SG55, (syn=SO-55/G), SG3201, (syn=SO-3201/G), SHII, (syn=HII), SHV, (syn=SHV), SHX, SHX, SKII, (syn=K2), (syn=KII), (syn=SII), (syn=SsII), (syn=II), SKIV, (syn=SIVb), (syn=SsIV), (syn=IV), SKIVa, (syn=SIVab), (syn=SsIVa), (syn=IVa), SKV, (syn=K4), (syn=KV), (syn=SV), (syn=SsV), (syn=V), SKX, (syn=K9), (syn=KX), (syn=SX), (syn=SsX), (syn=X), STV, (syn=T35), (syn=35-50-R), STVIII, (syn=T8345), (syn=8345-SO—S-R), W1, (syn=D8), (syn=FSD8), W2a, (syn=D2A), (syn=FS2a), DD-2, Sf6, FS1, (syn=F1), SF6, (syn=F6), SG42, (syn=SO-42/G), SG3203, (syn=SO-3203/G), SKF12, (syn=SsF12), (syn=F12), (syn=F12), STII, (syn=1881-SO-R), γ66, (syn=gamma 66a), (syn=Ssy66), φ2, Bll, DDVII, (syn=DD7), FSD2b, (syn=W2B), FS2, (syn=F2), (syn=F2), FS4, (syn=F4), (syn=F4), FS5, (syn=F5), (syn=F5), FS9, (syn=F9), (syn=F9), F11, P2-SO-S, SG36, (syn=SO-36/G), (syn=G36), SG3204, (syn=SO-3204/G), SG3244, (syn=SO-3244/G), SHI, (syn=HI), SHVII, (syn=HVII), SHIX, (syn=HIX), SHXI, SHXII, (syn=HXII), SKI, KI, (syn=SI), (syn=SsI), SKVII, (syn=KVII), (syn=SVII), (syn=SsVII), SKIX, (syn=KIX), (syn=SIX), (syn=SsIX), SKXII, (syn=KXII), (syn=SVII), (syn=SsXII), STI, SIII, STIII, STIV, STVII, S70, S206, U2-SO-S, 3210-SO-S, 3859-SO-S, 4020-SO-S, φ3, φ5, φ7, φ8, φ9, φ10, φ11, φ13, φ14, φ18, SHIII, (syn=HIII), SHXI, (syn=HXI) and SXI, (syn=KXI), (syn=SXI), (syn=SsXI), (syn=XI).

Bacteria of the genus Staphylococcus are infected by the following phage: A, EW, K, Ph5, Ph9, Ph10, Ph13, P1, P2, P3, P4, P8, P9, P10, RG, SB-1, (syn=Sb-1), S3K, Twort, φSK311, φ812, 06, 40, 58, 119, 130, 131, 200, 1623, STC1, (syn=stc1), STC2, (syn=stc2), 44AHJD, 68, AC1, AC2, A6″C″, A9″C″, b581, CA-1, CA-2, CA-3, CA-4, CA-5, D11, L39x35, L54a, M42, N1, N2, N3, N4, N5, N7, N8, N10, N11, N12, N13, N14, N16, Ph6, Ph12, Ph14, UC-18, U4, U15, S1, S2, S3, S4, S5, X2, Z1, φB5-2, φD, ω, 11, (syn=φ11), (syn=P11-M15), 15, 28, 28A, 29, 31, 31B, 37, 42D, (syn=P42D), 44A, 48, 51, 52, 52A, (syn=P52A), 52B, 53, 55, 69, 71, (syn=P71), 71A, 72, 75, 76, 77, 79, 80, 80a, 82, 82A, 83A, 84, 85, 86, 88, 88A, 89, 90, 92, 95, 96, 102, 107, 108, 111, 129-26, 130, 130A, 155, 157, 157A, 165, 187, 275, 275A, 275B, 356, 456, 459, 471, 471A, 489, 581, 676, 898, 1139, 1154A, 1259, 1314, 1380, 1405, 1563, 2148, 2638A, 2638B, 2638C, 2731, 2792A, 2792B, 2818, 2835, 2848A, 3619, 5841, 12100, AC3, A8, A10, A13, b594n, D, M12, N9, N15, P52, P87, S1, S6, Z4, φRE, 3A, 3B, 3C, 6, 7, 16, 21, 42B, 42C, 42E, 44, 47, 47A, 47C, 51, 54, 54x1, 70, 73, 75, 78, 81, 82, 88, 93, 94, 101, 105, 110, 115, 129/16, 174, 594n, 1363/14, 2460 and NN-Staphylococcus (1).

Bacteria of the genus Streptococcus are infected by the following phage: EJ-1, NN-Streptococcus (1), a, C1, FLOThs, H39, Cp-1, Cp-5, Cp-7, Cp-9, Cp-10, AT298, A5, a10/J1, a10/J2, a10/J5, a10/J9, A25, BT11, b6, CA1, c20-1, c20-2, DP-1, Dp-4, DTI, ET42, e10, FA101, FEThs, FK, FKK101, FKL10, FKP74, FK11, FLOThs, FY101, f1, F10, F20140/76, g, GT-234, HB3, (syn=HB-3), HB-623, HB-746, M102, O1205, φO1205, PST, P0, P1, P2, P3, P5, P6, P8, P9, P9, P12, P13, P14, P49, P50, P51, P52, P53, P54, P55, P56, P57, P58, P59, P64, P67, P69, P71, P73, P75, P76, P77, P82, P83, P88, sc, sch, sf, Sfi11, (syn=SFi11), (syn=φSFi11), (syn=ΦSfi11), (syn=φSfi11), sfi19, (syn=SFi19), (syn=φSFi19), (syn=φSfi19), Sfi21, (syn=SFi21), (syn=φSFi21), (syn=φSfi21), STG, STX, st2, ST2, ST4, S3, (syn=φS3), s265, Φ17, φ42, Φ57, φ80, φ81, φ82, φ83, φ84, φ85, φ86, φ87, φ88, φ89, φ90, φ91, φ92, φ93, φ94, φ95, φ96, φ97, φ98, φ99, φ100, φ101, φ102, φ227, Φ7201, ω1, ω2, ω3, ω4, ω5, ω6, ω8, ω10, 1, 6, 9, 10F, 12/12, 14, 17SR, 19S, 24, 50/33, 50/34, 55/14, 55/15, 70/35, 70/36, 71/ST15, 71/45, 71/46, 74F, 79137, 79/38, 80/J4, 80/J9, 80/ST16, 80/15, 80/47, 80/48, 101, 103/39, 103/40, 121/41, 121/42, 123/43, 123/44, 124/44, 337/ST17 and NN-Streptococcus (34).

Bacteria of the genus Treponema are infected by the following phage: NN-Treponema (1).

Bacteria of the genus Vibrio are infected by the following phage: CTXΦ, fs, (syn=s1), fs2, 1vpfs, Vf12, Vf33, VPIΦ, VSK, v6, 493, CP-TI, ET25, kappa, K139, LaboI,) XN-69P, OXN-86, O6N-21P, PB-1, P147, rp-1, SE3, VA-1, (syn=VcA-1), VcA-2, VcA-1, VPI, VP2, VP4, VP7, VP8, VP9, VP10, VP17, VP18, VP19, X29, (syn=29 d'Hérelle), 1, ΦHAWI-1, ΦHAWI-2, ΦHAWI-3, ΦHAWI-4, ΦHAWI-5, ΦHAWI-6, ΦHAWI-7, ΦHAWI-8, ΦHAWI-9, ΦHAWI-10, ΦHC1-1, ΦHC1-2, ΦHC1-3, ΦHC1-4, ΦHC2-1, ΦHC2-2, ΦHC2-3, ΦHC2-4, ΦHC3-1, ΦHC3-2, ΦHC3-3, ΦHD1S-1, ΦHD1S-2, ΦHD2S-1, ΦHD2S-2, ΦHD2S-3, ΦHD2S-4, ΦHD2S-5, ΦHDO-1, ΦHDO-2, ΦHDO-3, ΦHDO-4, ΦHDO-5, ΦHDO-6, ΦKL-33, ΦKL-34, ΦKL-35, ΦKL-36, ΦKW1H-2, ΦKWH-3, ΦKWH-4, ΦMARQ-1, ΦMARQ-2, ΦMARQ-3, ΦMOAT-1, ΦO139, ΦPEL1A-1, ΦPEL1A-2, ΦPEL8A-1, ΦPEL8A-2, ΦPEL8A-3, ΦPEL8C-1, ΦPEL8C-2, ΦPEL13A-1, ΦPEL-13B-1, ΦPEL13B3-2, ΦPEL13B-3, ΦPEL13B-4, ΦPEL13B-5, ΦPEL13B-6, ΦPEL13B-7, ΦPEL13B-8, ΦPEL13B-9, ΦPEL13B-10, φVP143, φVP253, Φ16, φ138, 1-11, 5, 13, 14, 16, 24, 32, 493, 6214, 7050, 7227, II, (syn=group 11), (syn=φ2), V, VIII, NN-Vibrio (13), KVP20, KVP40, nt-1, 06N-22P, P68, e1, e2, e3, e4, e5, FK, G, J, K, nt-6, Ni, N2, N3, N4, N5, O6N-34P, OXN-72P, OXN-85P, OXN-100P, P, Ph-1, PL163/10, Q, S, T, φ92, 1-9, 37, 51, 57, 70A-8, 72A-4, 72A-10, 110A-4, 333, 4996, I, (syn=group I), III, (syn=group III), VI, (syn=A-Saratov), VII, IX, X, NN-Vibrio (6), pA1, 7, 7-8, 70A-2, 71A-6, 72A-5, 72A-8, 108A-10, 109A-6, 109A-8, 110A-1, 110A-5, 110A-7, hv-1, OXN-52P, P13, P38, P53, P65, P108, P111, TP1, VP3, VP6, VPI2, VP13, 70A-3, 70A-4, 70A-10, 72A-1, 108A-3, 109-B1, 110A-2, 149, (syn=φ149), IV, (syn=group IV), NN-Vibrio (22), VP5, VP 1, VP15, VP16, α1, α2, α3a, 3b, 353B and NN-Vibrio (7).

Bacteria of the genus Yersinia are infected by the following phage: H, H-i, H-2, H-3, H-4, Lucas 110, Lucas 303, Lucas 404, YerA3, YerA7, YerA20, YerA41, 3/M64-76, 5/G394-76, 6/C753-76, 8/C239-76, 9/F18167, 1701, 1710, PST, 1/F2852-76, D'Hérelle, EV, H, Kotljarova, PTB, R, Y, YerA41, φYerO3-12, 3, φA1122, 4/C1324-76, 7/F783-76, 903, 1/M6176 and Yer2AT.

In another embodiment, the methods are practiced using a combination of at least 2, at least 3, at least 4, at least 5, at least 6, at least 7, at least 8, at least 9 or a greater number of the aforementioned phages. One skilled in the art recognizes that the efficiency of transformation may be manipulated, e.g., enhanced or suppressed, depending on the particular combination of phages that are employed.

In particular, bacteria species (and corresponding, host-specific bacteriophages) include Aeromonas hydrophila (PM2), Bacillus anthracis (Gamma), Bacillus subtilus (SPP 1), Bordetella pertussis (See Pereversev et al. Zh Mikrobiol 5:54-57, 1981), Borrelia burgdorferi (φBB-1, see Eggers et al., J Bacteriol 183:4771-4778, 2001), Brucella abortus (TB; 212; 371), Campylobacter jejuni (φ4, φC), Clostridium perfringes (φ3626), Enterococcus faecalis (φFC1), Enterococcus faecium (ENB6), Escherichia coli (P1; Ti; T3, T4, T5; T7, KH1, φV10; lambda; φ20; mu), Klebsiella pneumoniae (60; 92), Listeria monocytogenes (A511, A 118; 243; H387; 2389; 2671; 2685; 4211), Mycobacterium leprae (mycobacteriophage, L5), Mycobacterium tuberculosis (LG; DSGA), Pseudomonas aeruginosa (E79, G101; B3; pp. 7), Salmonella anatum (E5), Salmonella bovismorbificans (98), Salmonella choleraesuis (102), Salmonella enteritidis (L; P22; 102; FO; IRA; φ8), Salmonella Newington (E34), Salmonella schottmulleri (31; 102; F0; 14), Salmonella typhi (163; 175; Vii; ViVI; 8; 23; 25; 46; 175; F0), Serratia marcescens (S24VA), Shigella dysenteriae (φ80; P2; 2; 37), Shigella flexneri (Sf6), Staphylococcus aureus (K; P1; P14; UC18; 15; 17; 29; 42D; 47; 52; 53; 79; 80; 81; 83A; 92; Twort, φ11), Streptococcus pneumoniae (Dp-1; Cp-1; HB-3; EJ-1; MM1; VO1), Streptococcuspyogenes (φX240; 1A; 1B; T12; 12/12; 113; 120; 124; P58; H4489a), Vibrio cholerae (138; 145; 149; 163), and Yersinia pestis (A1122; R; Y; P1). Additional information is provided in U.S. Patent Publication No. 2009-0155768.

In particular, Tables 1-3 provide representative examples of particular host-specific phages and the hosts to which they are specific for, including, the receptors through which they mediate their actions. See also Bertozzi et al., FEMS Microbiology Letters, 363, 1-11, 2016.

TABLE 1
Receptors in the cell wall of Gram-positive bacteria. Host names are ordered
alphabetically.

| Phages | Family | Main host | Receptor(s) |
|---|---|---|---|
| γ | Siphoviridae | Bacillus | Membrane surface-anchored protein |
| | | anthracis | phage receptor (GamR) |
| SPP1 | Siphoviridae | Bacillus subtilis | Glucosyl residues of |
| | | | poly(glycerophosphate) on WTA |
| φ29 | Podoviridae | Bacillus subtilis | Cell WTA (primary receptor) |
| Bam35 | Tectiviridae | Bacillus | N-acetyl-muramic acid (MurNAc) |
| | | thuringiensis | |
| LL-H | Siphoviridae | Lactobacillus | Glucose moiety of LTA |
| | | delbrueckii | |
| B1 | Siphoviridae | Lactobacillus | Galactose component of the wall |
| | | plantarum | |
| B2 | Siphoviridae | Lactobacillus | Glucose substituents in in teichoic |
| | | plantarum | acid |
| 5, 13, c2, h, ml3, kh, L | Siphoviridae | Lactococcus | Rhamnose moieties in the cell wall |
| | | lactis | |
| ϕLC3, TP901erm, | Siphoviridae | Lactococcus | Cell wall polysaccharides |
| TP901-1 | | lactis | |
| p2 | Siphoviridae | Lactococcus | Cell wall saccharides |
| | | lactis | |
| A511 | Myoviridae | Listeria | Peptidoglycan (murein) |
| | | monocytogenes | |
| A118 | Siphoviridae | Listeria | Glucosaminyl and rhamnosyl |
| | | monocytogenes | components |
| A500 | Siphoviridae | Listeria | Glucosaminyl residues |
| | | monocytogenes | |
| ϕ812, ϕK | Myoviridae | Staphylococcus | Anionic backbone of WTA |
| | | aureus | |
| 52A | Siphoviridae | Staphylococcus | O-acetyl group from the 6-position |
| | | aureus | |
| W, ϕ13, ϕ47, ϕ77, | Siphoviridae | Staphylococcus | N-acetylglucosamine (GlcNAc) |
| ϕSa2m | | aureus | glycoepitope |
| ϕSLT | Siphoviridae | Staphylococcus | Poly(glycerophosphate) moiety of |
| | | aureus | LTA |

TABLE 2
Receptors in the cell wall of Gram-negative bacteria. Host names are ordered
alphabetically.

| Phages | Family | Main host | Receptor(s) |
|---|---|---|---|
| φCr30 | Myoviridae | Caulobacter | Paracrystalline surface (S) protein |
| | | crescentus | |
| 434 | Siphoviridae | Escherichia coli | Protein Ib (OmpC) |
| BF23 | Siphoviridae | Escherichia coli | Protein BtuB (vitamin B12 |
| | | | receptor) |
| K3 | Myoviridae | Escherichia coli | Protein d or 3A (OmpA) with LPS |
| K10 | Siphoviridae | Escherichia coli | Outer membrane protein LamB |
| Me1 | Myoviridae | Escherichia coli | Protein c (OmpC) |
| Mu G(+) | Myoviridae | Escherichia coli | Terminal Glcα-2Glcα1- or |
| | | | GlcNAcα1-2Glcα1- of LPS |
| Mu G(−) | Myoviridae | Escherichia coli | Terminal glucose with a βl,3 |
| | | | glycosidic link |
| M1 | Myoviridae | Escherichia coli | Protein OmpA |
| Ox2 | Myoviridae | Escherichia coli | Protein OmpA |
| ST-1 | Myoviridae | Escherichia coli | Terminal Glcα-2Glcα1- or |
| | | | GlcNAcα1-2Glcα1- of LPS |
| TLS | Siphoviridae | Escherichia coli | Antibiotic efflux protein TolC |
| TuIa | Myoviridae | Escherichia coli | Protein Ia (OmpF) with LPS |
| TuIb | Myoviridae | Escherichia coli | Protein Ib (OmpC) with LPS |
| TuII* | Myoviridae | Escherichia coli | Protein II* (OmpA) with LPS |
| T1 | Siphoviridae | Escherichia coli | Proteins TonA (FhuA) |
| T2 | Myoviridae | Escherichia coli | Protein Ia (OmpF) with LPS |
| T3 | Podoviridae | Escherichia coli | Glucosyl-α-1,3-glucose terminus |
| | | | of rough LPS |
| T4 | Myoviridae | E. coli K-12/E. coli | Protein O-8 (OmpC) with LPS |
| | | B | |
| T4 | Myoviridae | E. coli B | Glucosyl-α-1,3-glucose terminus |
| | | | of rough LPS |
| T5 | Siphoviridae | Escherichia coli | Polymannose sequence in the O- |
| | | | antigen |
| T6 | Myoviridae | Escherichia coli | Outer membrane protein Tsx |
| T7 | Podoviridae | Escherichia coli | LPS |
| U3 | Microviridae | Escherichia coli | Terminal galactose residue in LPS |
| λ | Siphoviridae | Escherichia coli | Protein LamB |
| ϕX174 | Microviridae | Escherichia coli | Terminal galactose in rough LPS |
| ϕ80 | Siphoviridae | Escherichia coli | Proteins FhuA and TonB |
| PM2 | Corticoviridae | Pseudoalteromonas | Sugar moieties on the cell surface |
| E79 | Myoviridae | Pseudomonas | Core polysaccharide of LPS |
| | | aeruginosa | |
| JG004 | Myoviridae | Pseudomonas | LPS |
| | | aeruginosa | |
| φCTX | Myoviridae | Pseudomonas | Core polysaccharide of LPS |
| | | aeruginosa | |
| φPLS27 | Podoviridae | Pseudomonas | Galactosamine-alanine region of |
| | | aeruginosa | LPS |
| ϕ13 | Cystoviridae | Pseudomonas | Truncated O-chain of LPS |
| | | syringae | |
| ES18 | Cystoviridae | Salmonella | Protein FhuA |
| Gifsy-1, Gifsy-2 | Siphoviridae | Salmonella | Protein OmpC |
| SPC35 | Siphoviridae | Salmonella | BtuB (main receptor) and O12- |
| | | | antigen (asssiting receptor) |
| SPN1S, SPN2TCW, | Podoviridae | Salmonella | O-antigen of LPS |
| SPN4B, SPN13U | | | |
| SPN6TCW, | Podoviridae | Salmonella | O-antigen of LPS |
| SPN8TCW, | | | |
| SPN9TCW | | | |
| SPN7C, SPN9C, | Siphoviridae | Salmonella | Protein BtuB |
| SPN10H, SPN12C | | | |
| SPN14, SPN17T, | Siphoviridae | Salmonella | Protein BtuB |
| SPN18 | | | |
| vB_SenM-S16 (S16) | Myoviridae | Salmonella | Protein OmpC |
| Ω8 | Podoviridae | Escherichia coli | α-1,3-mannosyl linkages |
| c341 | Podoviridae | Salmonella | O-acetyl group in the |
| | | | mannosylrhamnosyl-O- |
| | | | acetylgalactose |
| P22 | Podoviridae | Salmonella | O-acetylgalactose |
| € 34 | Podoviridae | Salmonella | [-β-Gal-Man-Rha-] polysaccharide |
| Sf6 | Podoviridae | Shigella | Rha II 1-α-3 Rha III linkage |

TABLE 3
Receptors in bacterial complexes other than cell walls. Host names are ordered
alphabetically.

| Phages | Family | Main host | Receptor(s) |
|---|---|---|---|
| SPN2T, SPN3C, | Siphoviridae | Salmonella | Flagellin protein FliC |
| SPN8T, SPN9T | | | |
| SPN11T, SPN13B, | Siphoviridae | Salmonella | Flagellin protein FliC |
| SPN16C | | | |
| SPN4S, SPN5T, | Siphoviridae | Salmonella | Flagellin proteins FliC or FljB |
| SPN6T, SPN19 | | | |
| iEPS5 | Siphoviridae | Salmonella | Flagellal molecular ruler protein |
| | | | FliK |
| ϕCbK, ϕCb13 | Siphoviridae | Caulobacter | Pili portals on the cell pole |
| | | crescentus | |
| Fd, Ff, f1, M13 | Inoviridae | Escherichia coli | F pilus followed by TolQRA |
| | | | complex in membrane |
| PRD1 | Tectiviridae | Escherichia coli | Mating pair formation (Mpf) |
| | | | complex |
| ϕ6 | Cystoviridae | Pseudomonas | Sides of the type IV pilus |
| MPK7 | Podoviridae | Pseudomonas | Type IV pili (TFP) |
| | | aeruginosa | |
| MP22 | Siphoviridae | Pseudomonas | Type IV pili (TFP) |
| | | aeruginosa | |
| DMS3 | Siphoviridae | Pseudomonas | Type IV pili (TFP) |
| | | aeruginosa | |
| 29 | Podoviridae | Escherichia coli | β-D-glucosido-(1-3)-D-glucoronic |
| | | | acid bonds in capsule |
| K11 | Podoviridae | Klebsiella | β-D-glucosyl-(1-3)-β-D- |
| | | | glucuronic acid linkages |
| Vi I | Myoviridae | Salmonella | Acetyl groups of the Vi |
| | | | exopolysaccharide capsule |
| Vi II | Siphoviridae | Salmonella | Acetyl groups of the Vi |
| | | | exopolysaccharide capsule |
| Vi III, Vi IV, Vi V, Vi | Podoviridae | Salmonella | Acetyl groups of the Vi |
| VI, Vi VII | | | exopolysaccharide capsule |

### D. Bacteriophage Packaging Sites

A bacteriophage packaging site is a specific DNA sequence on the phage genome for genome packaging into the virion. A plasmid is engineered to contain a phage packaging site so that plasmid is packaged into the transducing particles. Host-specific bacteriophages (and their packaging sites) include but are not limited to SPP1 (SPP1 pac site), P1 (P1 pac site), T1 (T1 pac site), T7 (T7 concatemer junction), lambda (cos site), mu (mu pac site), P22 (P22 pac site), φ8 (φ8 pac site), Sf6 (Sf6 pac site), 149 (149 pac site), and A1122 (A1122-concatamer junction). For most bacteriophages, the packaging site is termed the pac site. In some cases, the packaging site is referred to as a concatemer junction (e.g. T7 concatemer junction). In every case, the packaging site is different from the naturally-occurring adjacent sequences in the bacteriophage genome.

For some bacteriophages, the packaging site may be unknown. In these cases, pac sites can be determined by taking advantage of the property that plasmids containing a functional bacteriophage pac site are packaged. For example, the DNA sequences necessary for packaging of bacteriophage λ were determined by incorporating small restriction fragments of the λ phage genomic DNA into a plasmid (Hohn et al., PNAS USA 80:7456-7460, 1983). Following introduction into an in vivo packaging strain, the efficiency of packaging/transduction was quantitatively assessed. Using a similar strategy, the pac sites for a number of bacteriophages have been determined: λ (Miwa et al., Gene 20:267-279, 1982); Mu (Croenen et al., Virology 144:520-522, 1985); filamentous bacteriophages including f1, fd, M13, and Ike (Russel et al., J Virol., 63:3284-3295, 1989); P22 (Petri et al., Gene 88:47-55, 1990; Wu et al., Mol. Microbiol 45:1631-1646, 2002); T7 (Chung et al., J Mol Biol 216:927-938, 1990), and T3 (Hashimoto et al., Virology 187:788-795, 1992).

Embodiments of the methods include bacteriophage packaging sequences and functional fragments thereof. These nucleic acid embodiments can be for example, at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500, 550, 600, 650, 700, 750, 800, 850, and 900 nucleotides in length so long as the nucleotide fragment can mediate packaging of plasmid DNA into bacteriophage capsids (as judged by its ability to mediate packaging and thereby produce functional transducing particles). The nucleic acids that comprise the bacteriophage packaging sites or fragments thereof are incorporated into the plasmids.

### E. Marker Gene Constructs

Gene technology is widely used to monitor cellular gene expression (Naylor et al., Biochem Pharm 58:749-757, 1990). Preferably, the marker molecule is a gene which encodes a detectable product, e.g., a protein or enzyme. Particularly preferably, the marker is molecule that is not natively expressed by the phage, a human, or the bacteria. For example, the marker may be a heterologous eukaryotic protein, a protein of different bacterial species, or a viral protein. In some embodiments, marker is a plant-based enzyme or non-coding RNA. In some embodiments, the marker is a gene or a product encoded by a gene, wherein the gene is derived from a non-human animal.

In one embodiment, the marker is a hydrolytic enzyme.

The present methods comprise, in part, on determining the presence (or absence) or level (e.g., concentration) or activity (e.g., enzyme activity) of at least one marker or indicator in a sample. The term “marker” or “indicator”, as it is used herein, refers to a nucleotide sequence that encodes for a nucleic acid (e.g., mRNA), peptide or protein that permits determination or confirmation that the vector has been transfected or transduced correctly, and that its sequences are correctly expressed. A marker may also refer to the nucleic acid, peptide, protein, or enzyme that is produced by expression of a heterologous gene in a recombinant or engineered phage. The marker may be a nucleotide sequence encoding for a protein or a gene encoding for antibiotic resistance, used to select the cells that carry the vector. As used herein, the term “detecting the presence of at least one marker” includes determining the presence of each marker of interest by using any quantitative or qualitative assay known to one of skill in the art. In certain instances, qualitative assays that determine the presence or absence of a particular trait, variable, genotype, and/or biochemical or serological substance (e.g., protein or antibody) are suitable for detecting each marker of interest. In certain other instances, quantitative assays that determine the presence or absence of DNA, RNA, protein, antibody, or activity are suitable for detecting each marker of interest. As used herein, the term “detecting the level of at least one marker” includes determining the level of each marker of interest by using any direct or indirect quantitative assay known to one of skill in the art. In certain instances, quantitative assays that determine, for example, the relative or absolute amount of DNA, RNA, protein, antibody, or activity are suitable for detecting the level of each marker of interest. One skilled in the art will appreciate that any assay useful for detecting the level of a marker is also useful for detecting the presence or absence of the marker.

In some embodiments, the marker is a reporter molecule that can signify its presence, e.g., via its luminescent properties or its ability to conduct enzymatic reactions. In another embodiment, the marker binds to a reporter molecule to signify the level or activity of the marker.

(i) Reporters

In one embodiment, the reporter molecule is a gene, referred to as a reporter gene, that encodes for expression of a detectable protein. Commonly used reporter genes include chloramphenicol acetyltransferase (CAT), 13-galactosidase, luciferase, alkaline phosphatase, and green fluorescent protein (GFP). In general, reporter genes have the advantage of low background activity and sensitive signal detection following gene expression. For example, the development of luciferase and GFP as non-invasive markers of gene expression, combined with ease of detection using sensitive charge-coupled device (CCD) imaging cameras and fluorescence microscopy, has allowed for temporal and spatial information about gene expression even at the single cell level.

A review of luciferase genes and their use as reporter genes provides a list of known luciferase genes, cDNAs, proteins, and corresponding GENBANK Accession numbers for Vibrio harveyi (Accession Nos. M10961 and AAA88685), Vibrio harveyi (Accession Nos. M10961 and AAA88686), Vibrio harveyi (Accession Nos. M28815 and AAA27531), Vibrio fischeri (Accession Nos. X06758 and CAA29931) Vibrio fischeri (Accession Nos. X06797 and CAA29932), Vibrio fischeri (Accession No. AF170104 (including variants thereof)), Photorhabdus luminescens (Accession No. M62917), Photinus pyralis (M15077 and AAA29795), Luciola cruciate (Accession Nos. M26194 and AAA29135), Vargula hilgendorfii (Accession Nos. E02749, M25666, and AAA30332), Aequorea victoria (Accession Nos. M16103, AAA27719, Ml11394, AAA27716, M16104, AAA27717, M16105, AAA27718, L29571, AAA27720, and E02319); Oplophorus gracilorostris (Accession Nos. AB030246, BAB13776, AB030245 and BAB13775); Renilla muelleri (Accession Nos. AY015988 and AAG54094); and Renilla reniformis (Accession Nos. M63501 and AAA29804). See, Greer et al., Luminescence 17:43-74, 2002). Greer also provides a large number of constructs and vectors that are useful for imaging (see Table 2, pp 48-52). These vectors are suitable for expression in Staphylococcus aureus, E coli and other bacteria. Among the known luciferases are the prokaryotic luciferases (Lux), and eukaryotic luciferases (Luc, Ruc and their regulatory proteins) both of which are commonly used in imaging of luciferase expression in living cells.

In another embodiment, the reporter molecule comprises a β-galactosidase reporter gene expressed in bacteria (Miller et al., Experiments in Molecular Genetics, Cold Spring Harbor Laboratory, Cold Spring Harbor, N.Y.; Sambrook et al., Molecular Cloning, Cold Spring Harbor Laboratory, Cold Spring Harbor, N.Y.). β-galactosidase activity expressed by bacterial colonies is detected by blue coloration on medium containing X-Gal (5-bromo-4-chloro-3-indolyl-β-D-galactopyranoside). Chloramphenicol acetyltransferase (CAT) is also suitable for use as a reporter gene in bacteria. CAT is encoded by a bacterial drug-resistance gene (Kondo et al., J Bacteriol 88:1266-1276). It inactivates chloramphenicol by acetylating the drug at one or both of its two hydroxyl groups. In a typical CAT assay, cell extracts are incubated in a reaction mix containing 14C- or 3H-labeled chloramphenicol and n-Butyryl Coenzyme A. CAT transfers the n-butyryl moiety of the cofactor to chloramphenicol. The reaction products are extracted with xylene and the n-butyryl chloramphenicol partitions mainly into the xylene phase, while unmodified chloramphenicol remains predominantly in the aqueous phase. Radiolabeled chloramphenicol that partitions into the xylene phase is measured using a scintillation counter.

Bacterial alkaline phosphatase encoded by phoA of E. coli is enzymatically active only when it has been transported across the cellular membrane into the periplasmic space (Gibson et al., Appl and Env Microbiol 68:928-932, 2002). This property has been exploited to engineer PhoA protein as a molecular sensor of subcellular location. Another bacterial alkaline phosphatase (PhoZ) derived from the Gram-positive bacterium Enterococcus faecalis (Lee et al., J Bacteriol 181:5790-5799, 1999) has been developed as a reporter that is highly active in Gram-positive bacteria (Granok et al., J Bacteriol 182:1529-1540, 2000; Lee et al., J Bacteriol 181:5790-5799, 1999). The alkaline phosphatase activity of PhoZ, like that of PhoA, is dependent on its export from the cytoplasm. In an alkaline phosphatase assay, alkaline phosphatase hydrolyzes substrates such as 4-nitrophenyl phosphate (4NPP) to yield a chromogen (e.g. 4-nitrophenol, 4NP).

Reporter genes allow for simpler manipulation procedures (e.g. reduced purification or cell lysis), they are adaptable to large-scale, high throughput screening measurements, and they are compatible with bacteria systems. Reporter genes can be either naturally occurring genes or those produced by genetic manipulation, such as recombinant DNA technology or mutagenesis. Reporter genes are nucleic acid segments that contain a coding region and any associated expression sequences such as a promoter, a translation initiation sequence, and regulatory sequences.

(ii) Promoters

In some embodiments, phage-derived promoters are linked to the marker or reporter gene. In some embodiments, a bacteria-specific promoter is used.

A reporter gene is typically linked to a promoter sequence that controls and directs synthesis of RNA. It will be appreciated by those of ordinary skill in the art that a promoter sequence may be selected from a large number of bacterial genes expressed by various bacterial species. The choice of promoter is made based on the target bacteria to be detected. For a review of strategies for achieving high-level expression of genes in E. coli, see Makrides et al., Microbiol Rev 60:512-538, 1996. An exemplary promoter sequence effective in E. coli is the T7 promoter, but any promoter or enhancer that is functional in prokaryotic cells may be used. Useful promoters include, but are not limited to, lac promoter (E. coli), trp promoter (E. coli), araBAD promoter (E. coli), lac hybrid promoter, (E. coli), trc hybrid promoter (E. coli), PL (X), SP6, and T7.

A promoter sequence is selected on the basis of its ability to achieve a detectable level of expression in the target pathogenic bacteria. In a preferred embodiment, the reporter gene is preferably coupled to a promoter obtained from the pathogenic bacterial host to be detected. A constitutive promoter will express the reporter at a constant rate regardless of physiological demand or the concentration of a substrate. Alternatively, it may be advantageous to use an inducible promoter to control the timing of reporter gene expression. For inducible promoters such as the lac and trp operons, expression is normally repressed and can be induced at a desired time. In the absence of lactose, the lac promoter is repressed by lac repressor protein. Induction can be achieved by the addition of lactose or IPTG, preventing the binding of repressor to the lac operator. Similarly, the lip promoter is negatively regulated by a tryptophan-repressor complex that binds to the trp operator. For the trp operon, gene expression can be induced by removing tryptophan or by adding β-indoleacrylic acid.

(iii) Bacteria-Specific Origins of Replication

Origins of replication used in the plasmids may be moderate copy number, such as colEl ori from pBR322 (15-20 copies per cell) or the R6K plasmid (15-20 copies per cell) or they may be high copy number, e.g. pUC oris (500-700 copies per cell), pGEM oris (300-400 copies per cell), pTZ oris (>1000 copies per cell) or pBluescript oris (300-500 copies per cell). The origins of replication may be functional in E. coli or in any other prokaryotic species such as Bacillus anthracis or Yersinia pestis.

Plasmid replication depends on host enzymes and on plasmid encoded and plasmid-controlled cis and trans determinants. For example, some plasmids have determinants that are recognized in almost all Gram-negative bacteria and act correctly in each host during replication initiation and regulation. Other plasmids possess this ability only in some bacteria (Kues et al., Microbiol Rev 53:491-516, 1989). Plasmids are replicated by three general mechanisms, namely theta type, strand displacement, and rolling circle (reviewed by Del Solar et al. Microbio and Molec Biol Rev 62:434-464, 1998).

For replication by the theta type mechanism, the origin of replication can be defined as (i) the minimal cis-acting region that can support autonomous replication of the plasmid, (ii) the region where DNA strands are melted to initiate the replication process, or (iii) the base(s) at which leading-strand synthesis starts. Replication origins contain sites that are required for interactions of plasmid and/or host encoded proteins. Plasmids undergoing theta type replication also include pPS10, RK2 (containing oriV), RP4, R6K (containing oriy), ColE1 and CoIE2. ColE1 is the prototype of a class of small multicopy plasmids that replicate by a theta-type mechanism. The origin of C61E1 replication spans a region of about 1 kb (Del Solar et al. 1998).

Examples of plasmids replicating by the strand displacement mechanism are the promiscuous plasmids of the IncQ family, whose prototype is RSF 1010. Members of this family require three plasmid-encoded proteins for initiation of DNA replication. These proteins promote initiation at a complex origin region, and replication proceeds in either direction by a strand displacement mechanism. The origin of replication has been defined as the minimal region able to support bidirectional replication when the RSF 110 replication proteins (RepA, RepB, and RepC) are supplied in trans by a second plasmid. The minimal ori region includes three identical 20-bp iterons plus a 174 bp region that contains a GC-rich stretch (28 bp) and an AT-rich segment (31 bp) (Del Solar et al. 1998).

Replication by the rolling circle (RC) mechanism is unidirectional, and is considered to be an asymmetric process because synthesis of the leading strand and synthesis of the lagging strand are uncoupled. Studies on the molecular mechanisms underlying RC replication have been done mainly with the staphylococcal plasmids pT181, pC221, pUB110, pC194, and with the streptococcal plasmid pMV158 and its Amob derivative pLS1. Other plasmids or phages that undergo RC replication include but are not limited to pS194, fd, φX174, pE194 and pFX2 (Del Solar et al. 1998).

Prokaryotes have a circular molecule of chromosomal DNA, typically with a single origin of replication. For example, the chromosomal origin of replication of E. coli and other bacteria is termed oniC. The present methods envision the use of origins of replication known in the art that have been identified from species-specific plasmid DNAs (e.g. ColE1, R1, pT181, and the like discussed herein above), from bacteriophages (e.g. φX174 and M13) and from bacterial chromosomal origins of replication (e.g. oriC).

(iv) Antibiotic Resistance Genes

The plasmid DNA of the transducing particles will optionally have an antibiotic resistance gene to facilitate molecular biology cloning of the plasmid and to allow for selection of bacteria transformed by plasmid. Antibiotic resistance genes are well known in the art and include but are not limited to ampicillin resistance (Ampr), chloramphenicol resistance (Cmr), tetracycline resistance (Tetr), kanamycin resistance (Kanr), hygromycin resistance (hyg or hph genes), and zeomycin resistance (Zeor). Preferably, the antibiotic resistant gene protects the bacteria from the antimicrobial or cytotoxic effect of a drug other than (or different from) the drug whose resistance or susceptibility is being tested. In another embodiment, the antibiotic resistant gene protects the bacteria from the antimicrobial or cytotoxic effect of a drug which is the same as the drug whose resistance or susceptibility is being tested.

### F. Methods of Making Transducing Particles

The transducing particles or recombinant phages used in the present methods are obtained by modifying a naturally-occurring bacteriophage to carry a gene capable of transforming the target bacteria to an easily recognizable phenotype, referred to hereinafter as the reporter gene. The transducing particle must be capable of specifically introducing the reporter gene into the target bacterial host in such a way that the bacterial host can express the gene function in a detectable manner. A large number of bacteriophages exist and may be selected for modification based on the desired host range and the ability of the bacteriophage to carry and transduce the gene of interest. In particular, the bacteriophage must be large enough to accommodate the reporter gene, the associated promoter region, the phage packaging site and any other DNA regions which may be included. Modified bacteriophages will usually retain the normal host range specificity of the unmodified bacteriophage, although some alteration in specificity would be acceptable so long as it does not affect the ability to identify the selected target bacteria.

The bacteriophages to be modified may be temperate or virulent, preferably being temperate. Modification of the bacteriophage results in a transducing particle that remains capable of introducing the reporter gene into a target bacterial host. The reporter gene is part of a plasmid or other self-replicating episomal unit which will be sustained and expressed in the infected host.

Transduction of the reporter gene may take place via transient expression (i.e., expression from a reporter gene which is not stably inherited by the cell) of the reporter gene. In such case, the DNA transduced by the bacteriophage may not survive intact through the entire test period. However, transcription of the reporter gene transduced by the phage will be sufficiently efficient before the DNA is degraded to ensure that the bacteria has assembled a functional reporter gene by the end of the test period. The bacteria can thus be detected by the assay even if the bacteria degrade the phage DNA.

Bacteriophages useful in the present methods may be obtained from microbiological repositories, such as the American Type Culture Collection, P.O. Box 1549, Manassas, Va., 20108, USA. Virulent bacteriophages are available as bacteria-free lysates, while lysogenic bacteriophages are generally available as infected host cells. Wild-type bacteriophage obtained from any source may be modified by conventional recombinant DNA techniques in order to introduce a desired reporter gene capable of producing the detectable phenotype of interest. Prior to introduction, the reporter gene of interest will be combined with a promoter region on a suitable gene cassette. The gene cassette may be constructed by conventional recombinant DNA techniques in a suitable host, such as E. coli. Both the reporter gene and the promoter region should be chosen to function in the target host, and the cassette may optionally include a second reporter gene, such as antibiotic resistance, heavy metal resistance, or the like, to facilitate in vitro manipulation.

The reporter gene (or genes, if not a single gene system) are capable of expressing a screenable phenotype in the target bacterial host. As used hereinafter, the phrase screenable phenotype is intended to mean a characteristic or trait which allows cells that express the phenotype to be distinguished from other cells which do not express the phenotype, even when all cells are growing and reproducing normally in a mixed culture. That is, detection of the characteristic or trait may be carried out while the infected target cells are present in mixed population of viable, usually proliferating non-target bacteria which do not express the phenotype. Preferably, the screenable phenotype will comprise a visually detectable trait, i.e., one that can be directly or indirectly observed in a mixed population of target and non-target cells. The phenotype will usually not be selectable, i.e., one which provides for survival or preferential growth under particular conditions (positive selection) or which provides for growth inhibition or killing under particular conditions. The method does not require that target bacteria present in the sample be isolated from or enriched relative to non-target bacteria which may be present in the sample because the trait will be observable when target bacteria comprise only a portion of the viable bacteria present.

The reporter gene can encode the screenable phenotype by itself or may be part of a multiple gene system encoding the phenotype, where other genes are present in or separately introduced to the host to be detected. For example, the transducing particle may carry the lacZa gene which requires a complementary lacZ3 gene or lacZAM15 deletion in the host for expression.

Suitable screenable phenotypes include bioluminescence, fluorescence, enzyme-catalyzed color production (e.g., using the enzyme alkaline phosphatase), and the like. Each of these phenotypes may be observed by conventional visualization techniques which provide the chemical reagents necessary to complete a signal producing reaction. In some embodiments, the screenable phenotype is a heterologous hydrolytic enzyme catalyzes the hydrolysis of a substrate into a detectable product such as a fluorophore or a chromogen. In some embodiments, the screenable phenotype is a heterologous, non-coding RNA, which can be amplified and detected by a probe that binds the resultant amplicon with specificity. In some embodiments, the heterologous, non-coding RNA can be detected by RT-HDA.

For the bacteriophage, it is possible to package the plasmid or the reporter gene cassette by attachment of the bacteriophage packaging site in a DNA construct with the plasmid or cassette. The packaging site may be obtained from the bacteriophage genome and cloned into the plasmid carrying the reporter gene, promoter region, and optional second reporter. The plasmid may then be transferred to a suitable bacterial host. The bacterial host will then produce transducing particles having the plasmid and/or marker gene cassette packaged within a bacteriophage coat capable of inserting the plasmid DNA into bacteria of its host range. The plasmid is transposed into the desired bacteriophage by simultaneous infection of a suitable host with both the plasmid and the bacteriophage. The host cells are incubated and the transducing particles are collected. A fraction of the phage will be carrying the plasmid. The transducing particles can be separated from the phage by conventional techniques.

The host-specific bacteriophage packaging sites are substantially isolated from sequences naturally occurring adjacent thereto in the bacteriophage genome. As used herein, the term “substantially isolated” with respect to bacteriophage packaging sites, means they that are not in their natural environment. That is, the packaging sites are not in a full-length, bacteriophage genomic nucleic acid sequence found in nature. The packaging sites may be isolated from the full length bacteriophage genomic sequence via experimental techniques, such as use of restriction endonuclease enzymes and cloning or amplification by the polymerase chain reaction. The packaging sites also may be produced synthetically.

A bacteriophage packaging site is a nucleic acid fragment devoid, in whole or part, of sequences normally associated with it in nature; or a sequence, as it exists in nature, but having heterologous sequences in association therewith. It is a fragment disassociated from the phage genome.

As used herein, the phrase “functional equivalents” in the context of bacteriophage packaging sites means packaging sites that function the same, qualitatively, as the wild type bacteriophage packaging sites. Thus, if an isolated bacteriophage packaging site directs packaging of DNA, a DNA fragment would be a functional equivalent if it also directs packaging of DNA in the same manner. Quantitative equivalence is not needed for a fragment to be a functional equivalent according to the method. Thus bacteriophage packaging sites that have nucleotide substitutions, deletions and/or additions can be functional equivalents of an isolated bacteriophage packaging site.

### G. Methods of Using Bacteriophages

The aforementioned embodiments may be practiced using transducing phage particles made up of fully intact phages or variants thereof comprising minimal structural elements to allow transduction of the particles into host cells. In some instances it will be possible to infect a biological sample and observe the alteration and phenotype directly, although in other cases it may be preferred to first prepare a mass culture of the bacteria present in the sample. Methods for obtaining samples and (if necessary) preparing mass culture will vary depending on the nature of the biological sample, and suitable techniques for preparing various sample types are described in detail in standard microbiology and bacteriology texts such as Bergey's Manual of Determinative Bacteriology (8^{th} ed.), Buchanan and Gibbons (eds.) Williams & Wilkens Co., Baltimore (1974); Manual of Methods for General Bacteriology, Gerhardt et al. (eds.), Am. Soc. Microbiology, Wash. (1981); and Manual of Clinical Microbiology (8th ed.), Patrick, R et al. (eds.), Am. Soc. Microbiology, Washington (2003).

The phage itself may be added to the sample in a variety of forms. It may be added in a dry state. The phage may be mixed or suspended into a liquid reagent mixture. It may be suspended in a vial to which the sample is added. It also may take any other suitable form. The phage added to the sample is sometimes herein referred to as “the parent phage.” Once contacted with bacteriophage, the sample is referred to as a phage exposed sample.

The phage exposed sample may be incubated for a predetermined time. Incubation may be for a sufficient time to allow production of the phage marker in infected target bacteria if present in the exposed sample. The phage exposed sample is in a condition that is conducive to phage infection of the target bacteria. This can be accomplished in a variety of ways. For example, the parent phage may be mixed into a reagent that, when added to the sample, results in a test sample conducive to infection. The sample may be prepared in many different ways to establish conditions conducive to phage infection.

Assuming there were target bacteria in the sample, the test sample will contain a phage marker. The parent phage infects the target bacteria by attaching themselves to cell walls of the target bacteria and injecting the viral nucleic acid to create infected bacteria. The recombinant bacteriophage marker gene is then abundantly expressed in the infected bacteria. If the bacteria are metabolically active, e.g., growing or dividing, then each progeny will contain additional copies of the marker gene (or be infected by the phage), thus, generating larger signals. In contrast, if the bacteria are quiescent or dead, then smaller signals are generated.

The marker may be analyzed via implementation of a plurality of processing steps. In one embodiment, the method involves lysing bacteria. In one embodiment, a microbial lysozyme is added to the bacteriophage exposed sample. In one embodiment, lysing involves adding chloroform to the bacteriophage exposed sample, treating the bacteriophage exposed sample with acid, or otherwise physically processing the bacteriophage exposed sample.

In contrast to other methods, production of progeny phage, rupturing of the host, release of progeny phage into the test sample and subsequent rounds of bacterial infection are not required. Moreover, while many prior art methods rely on detecting intact progeny phages, an embodiment of the present disclosure involves the detection of an overexpressed marker protein, which is not natively expressed by the bacteria or the phage or bacteria-infected host such as human. In other embodiments, the product of the marker gene may confer certain phenotype, e.g., antibiotic resistance or enhanced growth property, which may be functionally screened.

In one embodiment, the bacteriophage marker is an indirect indicator of the presence of target bacteria in the sample. Where the bacteriophage marker is a component of parent phage, the initial concentration of parent phage in the exposed sample may be controlled such that the background signal produced is undetectable in the assay. Thus, if no target bacteria are present in the sample, no infection occurs, no recombinant bacteriophage marker gene is expressed, and no new bacteriophage marker is synthesized. In one embodiment, a negative control is run using a control sample that is known to lack the target bacterial type in order to confirm that the bacteriophage used does not produce a background signal in the assay. Other aspects of the disclosure may provide for use of a negative control to identify a background signal that is distinguishable from any signal arising from an exposed sample comprising target bacteria.

In certain embodiments, once the biological sample has been prepared (with or without growth of a mass culture), it will typically be exposed to transducing particles under conditions which promote binding of the particles to the bacteria and injection of the genetic material, typically at a temperature which supports rapid growth of the bacteria (e.g., 35° C. to 40° C.) without agitation for a time sufficient to allow infection (e.g., 15 minutes to 120 minutes). Following infection, the cells are incubated to allow expression of the reporter gene and reporter gene expression is detected as described below.

The methods are applicable for homogenous isolates as well as heterogeneous bacterial samples, comprising, e.g., a plurality of species of bacteria. The term “plurality” means two or more units, e.g., species of bacteria, although the individual units need not be structurally and/or functionally different. In certain embodiments, the samples may be screened to provide a homogeneous bacterial population, e.g., using a particular nutritional media that is adapted to the particular population.

In contrast to conventional phage transduction techniques intended to produce homogeneous colonies of transduced bacterial cells, the methods do not require that the transduced bacteria be isolated in any way. Instead, the screenable phenotype, e.g., a visually observable trait, conferred by the reporter gene or a product thereof, can be detected in a non-selected portion of the biological sample where viable, usually proliferating, non-target bacteria will be present. The screening can occur without selection since there is no need to isolate the transduced bacteria.

In some embodiments, the method comprises analyzing a sample for the presence or absence of the marker nucleic acid or a product thereof. Suitable methods for the detection of marker nucleic acids or products thereof are known in the art, and can and will vary depending upon the nature of the sample.

In some embodiments, methods for determining susceptibility or resistance of bacteria to an antibiotic are provided, by carrying out the aforementioned antibiotic treatment, phage transformation and detection steps. These steps of antibiotic treatment and phage transformation may be carried out in any order or simultaneously. In one embodiment, the steps of antibiotic treatment and phage transformation are conducted sequentially. The term “sequentially” as used herein means that the steps are carried out in sequence, for example at an interval or intervals of minutes, hours, days or weeks. If appropriate the steps may be carried out in a regular repeating cycle. In another embodiment, the antibiotic treatment and phage transformation steps are carried out together, followed by the determination steps.

### H. Detection Methods

Methods of detecting a reporter gene or a product thereof may be indirect or direct. Indirect detection may comprise separating the reporter gene or a product thereof from other components in the sample, or concentrating reporter gene or a product thereof in the sample, followed by detection of reporter gene or a product thereof in the purified or concentrated sample. The reporter gene or product thereof may be detected in the liberated state (e.g., in the media containing phages) or in the bound form (e.g., contained inside bacteria either in the cytosol or integrated into the genome). In some embodiments, the reporter may be a protein having enzymatic activity, e.g., CAT activity or AP activity, as described previously. In such instances, the reporter activity is determined using enzymatic techniques. In some embodiments, the reporter molecule is a hydrolytic which catalyzes the hydrolysis of a substrate into a detectable molecule.

In one embodiment, the reporter protein or a fragment thereof is detected using mass spectrometry. In particular, techniques linking a chromatographic step with a mass spectrometry step may be used. Generally speaking, the presence of reporter protein or a fragment thereof may be determined utilizing liquid chromatography followed by mass spectrometry.

In some embodiments, the liquid chromatography is high performance liquid chromatography (HPLC). Non-limiting examples of HPLC may include partition chromatography, normal phase chromatography, displacement chromatography, reverse phase chromatography, size exclusion chromatography, ion exchange chromatography, bioaffinity chromatography, aqueous normal phase chromatography or ultrafast liquid chromatography. In one embodiment, the liquid chromatography may be ultrafast liquid chromatography.

In some embodiments, the mass spectrometry may be constant neutral loss mass spectrometry. In other embodiments, the mass spectrometry may be tandem mass spectrometry (MS/MS). In a different embodiment, the mass spectrometry may be matrix-assisted laser desorption/ionization (MALDI). In a specific embodiment, the mass spectrometry may be electrospray ionization mass spectrometry (ESI-MS).

In an exemplary embodiment, the method comprises liquid chromatography followed by tandem mass spectrometry. In a particularly exemplary embodiment, the method comprises liquid chromatography followed by tandem mass spectrometry as described in the examples. In another exemplary embodiment, the method comprises liquid chromatography followed by constant neutral loss mass spectrometry. In a particularly exemplary embodiment, the method comprises liquid chromatography followed by constant neutral loss mass spectrometry as described in the examples. In still another exemplary embodiment, the method comprises liquid chromatography followed by electrospray ionization mass spectrometry (ESI-MS).

In each of the above embodiments, the liquid chromatography followed by mass spectrometry may be used to determine the presence of the reporter protein or a fragment thereof in a sample, or the liquid chromatography followed by mass spectrometry may be used to determine the presence and quantity of the reporter protein or a fragment thereof in a sample. In preferred embodiments, the liquid chromatography followed by mass spectrometry may be used to determine the presence of the reporter protein or a fragment thereof in a sample.

In another embodiment, the reporter protein or a fragment thereof may be detected using cytometric techniques. Although methods for conducting cytometric measurements of cultured bacteria have been reported elsewhere (Martinez et al., Cytometry (1982) 3(2):129-33; Suller et al., J Antimicrob Chemother (1997) 40(1):77-83; and Roth et al., Appl Environ Microbiol (1997) 63(6):2421-31), they do not involve detection of phage reporter proteins. The cytometric detection methods can be adapted for both Gram-positive and Gram-negative bacteria, e.g., Escherichia coli (Martinez, supra), Bacillus cereus (Roth, supra), S. aureus (Suller et al., J Antimicrob Chemother (1997) 40(1):77-83), Staphylococcus epidermidis (Cohen et al., J Clin Microbiol (1989) 27(6): 1250-6), Streptococcus pyogenes (Cohen, supra), Klebsiella pneumoniae (Cohen, supra), Pseudomonas aeruginosa (Cohen, supra), P. stutzeri (Cohen, supra), Proteus mirabilis (Cohen, supra), and Enterobacter spp. (Cohen, supra).

In the aforementioned embodiments, the method makes use of host-specific recombinant or engineered phages. For example, a genetically modified φA1122 capable of infecting Yersinia pestis can be used for specific detection of Yersinia pestis. To detect multiple target bacterial types, one species of bacteriophage specific to each target bacterial type may be added to a single test sample, or individually to divisions thereof.

### I. Hydrolytic Enzymes

In some embodiments of the methods described herein, the recombinant or engineered bacteriophage may contain a gene for encoding a hydrolytic enzyme, which can be heterologous to the target bacterial species, to the bacteriophage, to a nonhuman animal, to a human, or combinations thereof. For the purposes of the present disclosure, a hydrolytic enzyme can be any enzyme that catalyzes the hydrolysis of one or more chemical bonds of a specific substrate. Examples of suitable substrates include, but are not limited to, compounds such as proteins, polypeptides, starches, fats, phosphate esters, and nucleic acids.

In some embodiments the hydrolytic enzyme can be selected from cellulases, cutinases, esterases, lipases, phosphoesterases, restriction endonucleases, and proteases. In some embodiments, the hydrolytic enzyme can be selected from the group consisting of collagenase, hyaluronidase, trypsin, chymotrypsin, pronase, elastase, DNase I, dispase, plasmin, bromelin, clostripain, thermolysin, neuraminidase, phospholipase, cholesterol esterase, subtilisin, papain, chymopapain, plasminogen activator, streptokinase, urokinase, fibrinolysin, serratiopeptidase, pancreatin, amylase, lysozyme, cathepsin-G, alkaline and acid phosphatases, esterases, decarboxylases, phospholipase D, P-xylosidase, β-D-fucosidase, thioglucosidase, β-D-galactosidase, a-D-galactosidase, a-D-glucosidase, β-D-glucosidase, β-D-glucuronidase, a-D-mannosidase, β-D-mannosidase, β-D-fructofuranosidase, β-D-glucosiduronase, and PMN leukocyte serine proteases. Other suitable hydrolytic enzymes will be apparent to those of skill in the art.

The hydrolytic enzyme encoded in the engineered or recombinant phage is preferably heterologous and not normally found in humans, bacteria, or the engineered or recombinant phage. The use of such heterologous genes and enzymes minimizes the potential for cross-reactivity of the test system of the presently disclosed methods with components native to the target bacterial species.

In some embodiments of the methods disclosed herein, the hydrolytic enzyme encoded by an engineered or recombinant phage is produced only upon specific bacteria-phage interaction. Thus, when the enzyme is produced it is a positive indication of the presence of the targeted bacterial species. As the engineered or recombinant phage is amplified and cell lysis occurs, the enzyme is released in solution whereupon it can interact with a substrate. Alternatively, the enzyme may be fused to a signal sequence to secrete the enzyme from the cell.

### J. Substrates

Substrates can be designed that will specifically interact with a chosen heterologous enzyme produced only upon interaction of a target bacterial species with an engineered or recombinant phage. The designed substrates can be included into a reagents formulation for culturing bacteria. Upon catalysis or hydrolysis of the substrate a detectable signal is produced. In some embodiments, the substrate is an intramolecularly quenched enzyme specific substrate. The intramolecularly quenched enzyme specific substrate can comprise a quenched signaling molecule such as a fluorophore or a chromogen, a quenching molecule (i.e., quencher), and a substrate linker. The substrate linker bridges the signaling molecule to the quencher, and holds the two in close proximity.

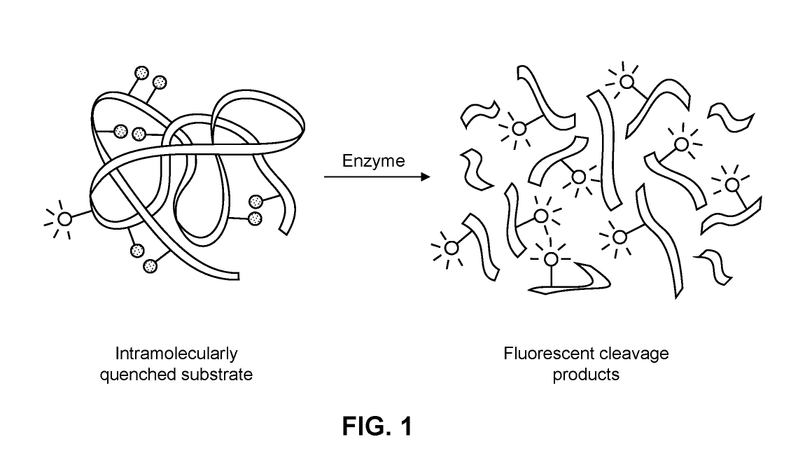

FIG. 1 depicts an illustration of one embodiment of an intramolecularly quenched enzyme specific substrate. In this illustration, one or more quenchers (dark circles) is linked to a signaling molecule (light circles with radiating lines extending outward) by a substrate linker (white ribbon shape). In some embodiments, one or more substrate linkers can link one or more quenchers with one or more signaling molecules. In some embodiments, a single signaling molecule is linked to a single quencher via a single substrate linker. In some embodiments, multiple signaling molecules are linked to a multiple quenchers via a single substrate linker.

In some embodiments, the quencher quenches fluorescence or a colorimetric signal only when in close proximity, for example about 3 nm to about 5 nm, to each other. Upon hydrolysis of the bridging substrate, the quencher and the signaling molecule are no longer bound in close proximity, and the quenching effect is reduced or removed. When the signaling molecule is a fluorophore or a chromogen, the unquenched signaling molecule can be detected by measuring a fluorescent or colorimetric signal. When the signal is above a predetermined threshold, the presence of the targeted bacterial species is indicated. In contrast, if the target bacterial species is not present, the phage will not amplify, and the signal will not be above a predetermined threshold.

In some embodiments, susceptibility of a target bacterial species can be detected by use of the presently disclosed enzyme specific substrates. In some embodiments, a target bacterial species incubated in the presence of an effective amount of an antibiotic or antimicrobial agent to which the target bacterial species is susceptible, will not amplify recombinant or engineered phage in an amount sufficient to produce a detectable signal or a detectable signal above a predetermined threshold. When the signal is not detected or is below the predetermined threshold, susceptibility to the antimicrobial agent is indicated. If a target bacterial species is resistant or not susceptible to the antibiotic or antimicrobial agent, then a detectable signal above the predetermined threshold will be produced. Thus, a negative or positive result can indicate the bacterial species' susceptibility to an antibiotic or antimicrobial agent.

Suitable signaling molecules will be apparent to those skilled in the art. For example a fluorophore and quencher combination can be selected from one of the pairings listed in Table 4 below

TABLE 4
Examples of fluorophore-quencher combinations

| | EXCITA- | | | ABSORB- |
|---|---|---|---|---|
| FLUOROPHORE | TION | EMISSION | QUENCHER | ANCE |
| Alexa 405 | 401 nm | 422 nm | DABCYL | 390-510 nm |
| FAM | 494 nm | 519 nm | BHQ-1 | 480-580 nm |
| Cy3 | 548 nm | 561 nm | BHQ-2 | 560-670 nm |
| Cy5 | 647 nm | 665 nm | BHQ-3 | 620-730 nm |

### K. Helicase Dependent Amplification (HDA)

When the marker is a gene encoding a heterologous non-coding RNA, a level or activity of the marker can be detected or measured using Reverse Transcriptase Helicase Dependent Amplification (RT-HDA). Procedures for the application of RT-HDA to the present methods are within the skill of person of ordinary skill in the art. Such methods are described in U.S. Pat. Nos. 7,282,328, 7,662,594, 7,829,284, 8,685,648, 9,303,288, 9,121,054, the contents are incorporated by reference herein in their entireties. Commercial methods and instrumentation using HDA are available, for example, the Solana system marketed by Quidel (San Diego, Calif.).

RT-HDA can amplify an engineered bacteriophage produced signal by a factor as high as about 10⁹, which makes RT-HDA amenable for use to detect markers at low levels. Upon infection of target cells, unique, stable self-splicing RNAs are produced which following cell lysis, can be detected by RT-HDA. RT-HDA can employ a predetermined mixture of reagents (reverse transcriptase, helicase, DNA polymerase, RNase HII, buffers, dNTPs and co-factors) which have been optimized for the detection of RNA templates. In some embodiments, primers specific for each non-coding RNA will be designed to span the intron junction using standard software. In some embodiments, the non-coding RNA is self-splicing. In addition, the RT-HDA may use a probe. In some embodiments, probes can be approximately 18-20 bases long, designed to overlap the splice junction sites of the non-coding RNA and/or a cDNA corresponding to the non-coding RNA (and thus be unique to the RNA, and be absent from the phage DNA), and harbor a fluorophore/quencher pair specific to each bacterial species that will emit fluorescence at a distinct wavelength. Detection and drug susceptibility analysis of cultured isolates can then be assessed.

In some embodiments, isothermal HDA is used. This technology uses DNA helicases to separate DNA strands during exponential amplification at a constant temperature of 65° C. As a result, instrumentation costs are significantly lower than PCR which require temperature cycling. Like PCR, HDA relies on 2 primers and can accommodate probe-based detection in a wide range of formats. Selected enzymes are able to amplify a target sequence in the presence of inhibitors commonly found in clinical specimens. HDA can amplify RNA faster than DNA because DNA helicase can unravel RNA:DNA and DNA:DNA duplexes, with both the reverse transcription and amplification reactions occurring simultaneously.

In an exemplary embodiment, a culture that has been transformed by an engineered phage having a heterologous gene coding a non-coding RNA is analyzed by RT-HDA in the following manner. Following infection, the non-coding RNA marker is expressed and released from the cell. The non-coding RNA can be a self-splicing RNA that comprises two or more spliced introns. The non-coding RNA is then transcribed using a reverse transcriptase to generate a cDNA amplicon. Helicase and other transcription aiding proteins can then unwind RNA:DNA and DNA:DNA double strands to aid in transcription and amplification. This cycle is repeated until a sufficient amount of amplicon has been produced. An intramolecularly quenched probe is used to detect the amplicon. The probe can comprise a quencher, a signaling molecule (e.g., a fluorophore or a chromogen), and a nucleic acid linker. The nucleic acid linker binds the signaling molecule and the quencher and acts as a bridge holding the quencher and the signaling molecule in close proximity (e.g., about 3 to about 5 nm). The linker is a nucleic acid designed to bind the amplicon at a site spanning the sequence location of the spliced intron junction of the non-coding RNA and/or the cDNA amplicon. The linker nucleic acid can have a single RNA base at the intron junction site to assist in rapid cleavage of the linker. Upon cleavage the signaling molecule and the quencher are no longer held in close proximity and quenching is reduced. RNases can be used to cleave a nucleic acid linker comprising one or more RNA bases. The signal effect of the signaling molecule can then be detected. In some embodiments, the signal is detected by fluorescence or colorimetric measurement.

In a first exemplary workflow according to one embodiment of a method described herein, a sample comprising bacteria is obtained. As described above, the sample can be from a subject, from a food item, from the environment, etc. The sample can be processed or treated, if needed or desired. An aliquot of the sample is incubated or cultured in the presence of an antibiotic and, optionally, an aliquot of the sample is incubated or cultured in the absence of the antibiotic. Simultaneously or sequentially, the sample aliquots are incubated or cultured with a recombinant or engineered phage specific for the bacteria in the sample. As described above, the engineered phage comprises a heterologous marker. Then the cultures generated by incubation with the antibiotic and the engineered phage are analyzed to determine presence or absence (quantitative or qualitative) of the marker and the result or data is reported.

In a second exemplary workflow according to another embodiment of a method described herein, a sample comprising a bacteria is obtained. The sample can be processed or treated, if needed or desired. Containers are prepared that contain a fluid medium with and without an antibiotic. Aliquots of sample are placed into each container, and mixed. Then, each container is incubated for a desired time at a desired temperature, and in this embodiment, each container is incubated for 2 hours at 35° C. The container can be mixed again after incubation, and then the engineered phage is introduced into the container, mixed, and then incubated to generate a secondary culture. An aliquot of the secondary culture is then analyzed for the presence or absence (quantitative or qualitative) of the heterologous marker.

The first and second workflows described above are exemplary for conducting the methods and assays described herein to screen candidate antibiotics for efficacy against a bacterial sample, a bacterial strain or a mix of bacterial strains. Generally, the method comprises contacting a bacterial sample with a test antibiotic to obtain a primary culture and with a vehicle lacking the test antibiotic to obtain a control primary culture; contacting a specific bacteriophage comprising a nucleic acid sequence that encodes for expression of a heterologous reporter gene with the primary culture and with the control primary culture, to obtain a first secondary culture that comprises bacteria treated with the test antibiotic(s) and a second secondary culture that comprises bacteria not treated with the test antibiotic(s); and detecting a level or activity of the reporter gene or a product thereof in the first and second secondary cultures, wherein a reduction (or absence) in the level or activity of the reporter gene or a product thereof in the first secondary culture compared to the second secondary culture indicates that the test compound is an antibiotic agent. The methods are also used for screening a single test antibiotic against a plurality of bacterial strains. The methods are also used for the minimum inhibitory concentration (MIC) of an antibiotic or candidate antibiotic and/or screening to determine the efficacy of a clinical antibiotic compound.

As used herein, the term “minimum inhibitory concentration” refers to the lowest concentration of an antibiotic that will inhibit the visible growth of a microorganism. The term also encompasses the lowest concentration of an antibiotic that effects bacterial cell death or inhibits cell wall repair using the methods and assays described herein. In one embodiment, the methods and assays described herein permit the determination of a minimum inhibitory concentration for an antibiotic or candidate antibiotic against a bacterial strain. In one embodiment, the minimum inhibitory concentration of an antibiotic can be determined by measuring a modulation in the response of the bacterial cells (e.g., uptake or extrusion of a reporter stain, change in morphology, change in metabolism, etc.) in a sample exposed to an antibiotic compared to the same bacterial cells in a sample not exposed to the antibiotic or in a sample exposed to different concentrations of the same antibiotic.

The minimum inhibitory concentration is a clinically relevant value indicating the minimum effective dose of an antibiotic to be administered to a subject to induce bacterial cell death and/or reduce at least one symptom of the bacterial-mediated disease. Clinically, the minimum inhibitory concentrations are used not only to determine the amount of antibiotic that a subject will receive but also to determine the preferred antibiotic to be used. A minimum inhibitory concentration can also be determined for a candidate antibiotic to permit e.g., efficacy determination and dosing information for clinical trials.

The present methods are useful for in vitro diagnosis as they permit determination of bacterial sensitivity to antibiotics and other bactericides. By performing a short incubation of the bacteria with an antibiotic or bactericide to be screened prior to exposure to the transducing particles, the metabolic activities of the cells will be halted and the alteration of the phenotype prevented. Such testing will be useful after the presence of the bacteria is initially confirmed using the transducing particles as described above. Antibiotics and bactericides which are determined to be lethal to the bacterial infection may then be employed for treatment of the subject. Such rapid and early detection of useful antibiotics and bactericides can be invaluable in treating severe bacterial infections.

In one embodiment, the diagnostic method may involve contacting a sample of a subject suffering from or suspected to be at risk of a bacterial disease with one or more recombinant phages as described herein; detecting and optionally quantitating the presence or absence of the marker expressed by the phage; correlating the presence of the marker to an etiological agent of the bacterial disease (e.g., S. aureus), thereby diagnosing the bacterial disease in the subject. By “subject” is meant any member of the phylum Chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term covers both adult and newborn individuals.

In certain embodiments, after a positive diagnosis of the bacterial disease is made, the subject may be optionally treated, managed, and followed-up in line with standard clinical procedures. For instance, a subject may be treated with an effective amount of a pharmaceutical agent, e.g., an antibiotic. For purposes of the present methods, an “effective amount” of an agent will be that amount which generates a response against the etiological agent, e.g., S. aureus, in a subject. In this regard, a subject suffering from pharyngitis may be treated with penicillin G benzathine and/or amoxicillin. If the subject is found to not respond to the treatment, the etiological agent may be analyzed for antibiotic resistance using the methods described above. If a positive identification as to the resistant strain is made, then the subject may be treated with a second antibiotic agent or a higher dosage of the antibiotic agent, or a combination of two or more antibiotic agents.

Similarly, the present methods are useful in detecting the presence of antibiotics, e.g., antibiotic residues in animal products. In this approach, an extract of the material to be analyzed is added to a culture of a bacterial strain sensitive to the antibiotic in question, and the mixture is incubated for a period predetermined to be sufficient to kill the strain if a given amount of antibiotic is present. At this point, transducing particles specific to the strain are added, and the assay as described herein is performed. If the given amount of antibiotic is present, the cells of the bacterial strain will be dead and the read-out will be negative (i.e., lack of luminescence in a luciferase assay). If the given amount of antibiotic is not present or lower than MIC, then bacteria will survive and the read-out will be positive (i.e., luminescence).

In a specific embodiment, a means is provided for assaying bacteria which have been previously rendered susceptible to transducing particles on a phage-specific basis. That is, in a first step, the target bacteria are modified, e.g., by transformation, so that they contain or express a cell-specific receptor for the bacteriophage of interest. In a second step, the modified (or tagged) bacteria are introduced into, or mixed into, a sample environment in which they are to be followed. The sample environment can be any setting where bacteria exist, including outdoors (e.g., soil, air or water); on living hosts (e.g., plants, animals, insects); on equipment (e.g., manufacturing, processing or packaging equipment); and in clinical samples. The bacteriophage assay as described herein is then conducted using bacteriophage specific for the introduced receptor, and the presence of the tagged bacteria can be monitored or quantified.

The aforementioned embodiments are further described in view of the following non-limiting examples.

### EXAMPLES

The structures, materials, compositions, and methods described herein are intended to be representative examples of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

### Example 1

### Construction of Recombinant Bacteriophage for Expression of Heterologous Marker

In this study, a molecule (marker) that is not naturally produced by target cells or by the phage vector or by the bacterium-infected host is prepared, followed by specific detection of the heterologous molecule (marker).

Construction of a Recombinant Bacteriophage:

A recombinant bacteriophage is constructed by inserting a DNA sequence encoding for the heterologous marker into a strongly expressed region of the phage genome downstream of the nucleic acid sequence encoding the capsid protein (cps) via homologous recombination mediated by a recombinant plasmid. A strong promoter, located upstream of cps, is selectively activated in the course of the expression of the bacteriophage genome following infection, producing many copies of the corresponding mRNA transcripts. Construction of the recombinant bacteriophage is accomplished using a fusion product of the nucleic acid encoding a reporter, having suitable translation signals (ribosome binding site, intermediate region, start codon). A representative method is described in Loessner et al., Appl. Environ. Microbiol., 62(4): 1133-40, 1996 and U.S. Pat. No. 5,824,468.

Electro-Transformation of the Plasmid Vector into an Electrocompetent E. coli K1 Strain (ATCC Strain 23503):

The strain is made electrocompetent by growing to an optical density (OD) of 0.8 at 37° C. in Luria-Bertani (LB) broth, followed by several washes in 15% glycerol. Electrotransformation is accomplished with a GENE PULSER available from Bio-Rad Labs (Hercules, Calif.).

Infection of the Transformed E. coli K1 Strain with Native Type Bacteriophage:

After infection of the host bacteria, at least a small number of the native bacteriophage will undergo homologous recombination with the portions of the capsid sequence flanking luxAB in the plasmid, thus transferring the luxAB to form recombinant phages. The transformed bacteria are grown to an optical density (OD) of 0.4 at 37° C. in LB-ampicillin media. Bacteriophage K1-5 is added at a multiplicity of infection (MOI) of approximately 1 bacteriophage per 10 bacteria, and the OD is monitored until lysis occurs. The lysate is collected by filtering through a 0.45 micron nitrocellulose membrane (available from Millipore Corp., Bedford, Mass.).

The lysate is plated and plaqued, using a serial dilution, onto wild type E. coli K1 (ATCC 23503) growing on LB solid agar with 50 gig per mL ampicillin and screened for recombinant K1-5 bacteriophage by assaying plaques for reporter activity. Confirmation that the recombinant bacteriophage has been generated containing the properly-integrated reporter gene sequence can be conducted by sequencing the phage genome. Sequencing is accomplished with the aid of a commercial sequencer (Commonwealth Biotechnologies, Richmond, Va.).

Finally, the expressed marker is detected by fluorescence or colorimetric measurement.

### Example 2

### Creation of Recombinant Bacteriophages Using DNA Transposition

A bacteriophage containing a heterologous reporter nucleic acid is constructed using the commercially available EZ::TN™ transposase system (Epicenter Technologies, Madison, Wis.) as described in Goryshin et al., J. Biol. Chem., 273(13):7367-7374, 1998. Then, the terminally ME-bound EZ::TN™ transposome is electrotransformed into an electrocompetent E. coli K1strain (ATCC strain 23503). The strain is made electrocompetent by growing to an optical density (OD) of 0.8 at 37° C. in LB media, followed by several washes in 15% glycerol. Electrotransformation is accomplished using the BIORAD GENE PULSER available from Bio-Rad Laboratories (Hercules, Calif.).

The transformed E. coli K1strain is infected with native type K1-5 bacteriophage. The transformed bacteria are grown to an OD of 0.4 at 37° C. in LB-ampicillin media. Bacteriophage K1-5 is added at a multiplicity of infection of approximately 1 bacteriophage per 10 bacteria, and the OD is monitored until lysis occurs. After infection of the transposome-electrotransformed host bacteria, at least a small number of the native K1-5 bacteriophage will receive the heterologous reporter gene by random transposition at an innocuous position that does not affect the plaque-forming ability of the phage. The lysate is collected by filtering through a 0.45 micron nitrocellulose membrane (available from Millipore Corp., Bedford, Mass.).

The lysate is screened for activity of the product of the reporter gene using standard methods, e.g., fluorimetry, colorimetry, etc.

### Example 3

### Screening Samples Obtained from a Subject Using Recombinant Bacteriophage

A subject (e.g., a human patient) exhibiting symptoms of bacterial infection (for example, fever, headache, abdominal pain, and nausea) is identified, and at least one of the following samples are collected from the subject: a 0.01 mL cerebrospinal fluid (CSF) sample, a 1.0 mL sputum sample, and a 1.0 mL blood sample. When all three of the samples are collected, each sample is diluted with 4.0 mL of LB broth, thus promoting growth of all bacteria present in the respective sample, and is incubated at 37° C. for 4 hours. After incubation, each sample is distributed by 100 μL aliquots into 30 wells of a 96-well plate. Aliquots of the blood sample are added to wells 1-30, aliquots of the CSF sample are added to wells 31-60, and aliquots of the sputum sample are added to wells 61-90. Wells 91-93 serve as positive controls, and wells 94-96 serve as negative controls. When simultaneously screening for susceptibility to one or more antimicrobial agents, some of the wells will have an antibiotic or a combination of antibiotics and some of the wells will have no antibiotic present.

The following five recombinant bacteriophage are obtained: K1-5::luxAB bacteriophage, which infects E. coli K1bacteria; EBN6::luxAB bacteriophage, which infects enterococcus bacteria; Twort::luxAB bacteriophage, which infects staphylococcus bacteria; Sp6::luxAB bacteriophage, which infects Salmonella bacteria; and RZh::luxAB bacteriophage, which infects streptococcus bacteria. The phages are obtained from another source or engineered in situ, using the protocol described in Example 1.

Recombinant bacteriophage suspension equivalent to about 3×10⁸ phages/mL is added to six individual wells of the groups of 30 wells corresponding to each of the three samples collected from the patient. An exemplary configuration of a 96-well plate for simultaneous identification and antimicrobial susceptibility of the three samples indicated above is provided in Table 5 below. Each engineered bacteriophage expresses a unique marker, each of which has a specific activity. With minor adjustments, this system is adapted for multiplex detection, e.g., wherein a plurality of samples is processed together or at the same time. The latter system is especially useful for the identification of a cohort of bacterial pathogens that are involved in the pathogenesis of a particular disease. For instance, pathogens associated with urinary tract infections (UTI), such as E. coli, Klebsiella, Enterobacter, Pseudomonas, Staphylococcus, Proteus, can all be identified and characterized using the multiplex array format.

TABLE 5
Example Configuration of a 96-Well Plate

| Well | Sample | Phage | Antibiotic |
|---|---|---|---|
| 1 | Blood | K1-5::luxAB | None |
| 2 | Blood | K1-5::luxAB | A β-lactam |
| 3 | Blood | K1-5::luxAB | A Fluoroquinolone |
| 4 | Blood | K1-5::luxAB | An Aminoglycoside |
| 5 | Blood | K1-5::luxAB | A Tetracycline |
| 6 | Blood | K1-5::luxAB | A Polymyxin |
| 7 | Blood | EBN6::luxAB | None |
| 8 | Blood | EBN6::luxAB | A β-lactam |
| 9 | Blood | EBN6::luxAB | A Fluoroquinolone |
| 10 | Blood | EBN6::luxAB | An Aminoglycoside |
| 11 | Blood | EBN6::luxAB | A Tetracycline |
| 12 | Blood | EBN6::luxAB | A Polymyxin |
| 13 | Blood | Twort::luxAB | None |
| 14 | Blood | Twort::luxAB | A β-lactam |
| 15 | Blood | Twort::luxAB | A Fluoroquinolone |
| 16 | Blood | Twort::luxAB | An Aminoglycoside |
| 17 | Blood | Twort::luxAB | A Tetracycline |
| 18 | Blood | Twort::luxAB | A Polymyxin |
| 19 | Blood | Sp6::luxAB | None |
| 20 | Blood | Sp6::luxAB | A β-lactam |
| 21 | Blood | Sp6::luxAB | A Fluoroquinolone |
| 22 | Blood | Sp6::luxAB | An Aminoglycoside |
| 23 | Blood | Sp6::luxAB | A Tetracycline |
| 24 | Blood | Sp6::luxAB | A Polymyxin |
| 25 | Blood | RZh::luxAB | None |
| 26 | Blood | RZh::luxAB | A β-lactam |
| 27 | Blood | RZh::luxAB | A Fluoroquinolone |
| 28 | Blood | RZh::luxAB | An Aminoglycoside |
| 29 | Blood | RZh::luxAB | A Tetracycline |
| 30 | Blood | RZh::luxAB | A Polymyxin |
| 31 | CSF | K1-5::luxAB | None |
| 32 | CSF | K1-5::luxAB | A β-lactam |
| 33 | CSF | K1-5::luxAB | A Fluoroquinolone |
| 34 | CSF | K1-5::luxAB | An Aminoglycoside |
| 35 | CSF | K1-5::luxAB | A Tetracycline |
| 36 | CSF | K1-5::luxAB | A Polymyxin |
| 37 | CSF | EBN6::luxAB | None |
| 38 | CSF | EBN6::luxAB | A β-lactam |
| 39 | CSF | EBN6::luxAB | A Fluoroquinolone |
| 40 | CSF | EBN6::luxAB | An Aminoglycoside |
| 41 | CSF | EBN6::luxAB | A Tetracycline |
| 42 | CSF | EBN6::luxAB | A Polymyxin |
| 43 | CSF | Twort::luxAB | None |
| 44 | CSF | Twort::luxAB | A β-lactam |
| 45 | CSF | Twort::luxAB | A Fluoroquinolone |
| 46 | CSF | Twort::luxAB | An Aminoglycoside |
| 47 | CSF | Twort::luxAB | A Tetracycline |
| 48 | CSF | Twort::luxAB | A Polymyxin |
| 49 | CSF | Sp6::luxAB | None |
| 50 | CSF | Sp6::luxAB | A β-lactam |
| 51 | CSF | Sp6::luxAB | A Fluoroquinolone |
| 52 | CSF | Sp6::luxAB | An Aminoglycoside |
| 53 | CSF | Sp6::luxAB | A Tetracycline |
| 54 | CSF | Sp6::luxAB | A Polymyxin |
| 55 | CSF | RZh::luxAB | None |
| 56 | CSF | RZh::luxAB | A β-lactam |
| 57 | CSF | RZh::luxAB | A Fluoroquinolone |
| 58 | CSF | RZh::luxAB | An Aminoglycoside |
| 59 | CSF | RZh::luxAB | A Tetracycline |
| 60 | CSF | RZh::luxAB | A Polymyxin |
| 61 | Sputum | K1-5::luxAB | None |
| 62 | Sputum | K1-5::luxAB | A β-lactam |
| 63 | Sputum | K1-5::luxAB | A Fluoroquinolone |
| 64 | Sputum | K1-5::luxAB | An Aminoglycoside |
| 65 | Sputum | K1-5::luxAB | A Tetracycline |
| 66 | Sputum | K1-5::luxAB | A Polymyxin |
| 67 | Sputum | EBN6::luxAB | None |
| 68 | Sputum | EBN6::luxAB | A β-lactam |
| 69 | Sputum | EBN6::luxAB | A Fluoroquinolone |
| 70 | Sputum | EBN6::luxAB | An Aminoglycoside |
| 71 | Sputum | EBN6::luxAB | A Tetracycline |
| 72 | Sputum | EBN6::luxAB | A Polymyxin |
| 73 | Sputum | Twort::luxAB | None |
| 74 | Sputum | Twort::luxAB | A β-lactam |
| 75 | Sputum | Twort::luxAB | A Fluoroquinolone |
| 76 | Sputum | Twort::luxAB | An Aminoglycoside |
| 77 | Sputum | Twort::luxAB | A Tetracycline |
| 78 | Sputum | Twort::luxAB | A Polymyxin |
| 79 | Sputum | Sp6::luxAB | None |
| 80 | Sputum | Sp6::luxAB | A β-lactam |
| 81 | Sputum | Sp6::luxAB | A Fluoroquinolone |
| 82 | Sputum | Sp6::luxAB | An Aminoglycoside |
| 83 | Sputum | Sp6::luxAB | A Tetracycline |
| 84 | Sputum | Sp6::luxAB | A Polymyxin |
| 85 | Sputum | RZh::luxAB | None |
| 86 | Sputum | RZh::luxAB | A β-lactam |
| 87 | Sputum | RZh::luxAB | A Fluoroquinolone |
| 88 | Sputum | RZh::luxAB | An Aminoglycoside |
| 89 | Sputum | RZh::luxAB | A Tetracycline |
| 90 | Sputum | RZh::luxAB | A Polymyxin |
| 91 | Blood Positive | None | None |
| 92 | CSF Positive | None | None |
| 93 | Sputum Positive | None | None |
| 94 | Blood Negative | None | None |
| 95 | CSF Negative | None | None |
| 96 | Sputum Negative | None | None |

### Example 4

### Analysis of the Wells for the Presence of the Marker

Each of the samples in the wells in Example 3 are further analyzed for the presence of the heterologous marker encoded by the recombinant phages applied therein. The presence of the marker is detected by use of a probe in each of the solutions of wells 1-96. The probe is added after incubation of the cultures for a predetermined amount of time. Alternatively, the cultures may be incubated in the presence of the probe.

### Analysis for Hydrolytic Enzyme Marker

Probes for hydrolytic enzymes are designed by linking a specific fluorophore with a specific quencher. For example, one probe combines FAM with BHQ-1, which has a fluorescence excitation wavelength of 494 nm and fluorescence emission wavelength of 519 nm. The quencher and the fluorophore are linked together in close proximity (e.g., 3-5 nm) by a linking peptide. When linked together by the peptide, the close proximity of the quencher and the fluorophore reduces the fluorescent properties of the fluorophore. Additional probes are created with specific fluorophore or chromogens with a corresponding quencher, and each probe has a unique linker selected from peptides, carbohydrates, and nucleic acids. Each unique linker will only be hydrolyzed if the corresponding hydrolytic enzyme marker specific to that linker is present in a given culture.

The probe is added to wells 1-93 of the 96-well plate, and incubation is allowed to continue for a predetermined amount of time. After the predetermined amount of time, the 96-well plate is introduced into a fluorimeter and the fluorescence and emission spectra are acquired in accordance with the specific fluorophores used in the probe. For the FAM probe, an excitation spectrum is acquired at 494 nm and an emission spectrum is acquired at 519 nm.

When the hydrolytic enzyme marker is present, the corresponding specific linker is hydrolyzed and fluorescence is detected. Detection of a threshold amount of fluorescence is indicative of the presence of the marker. No fluorescence is detected in wells 91-93, which do not have the marker, and wells 94-96, which do not have the marker or the probe. Wells having a target bacterial species present therein with no antibiotic present are transformed by the corresponding bacteriophage, and the hydrolytic enzyme marker is produced. The hydrolytic enzyme cleaves the substrate linker and quenching of the fluorophore is reduced or eliminated. Fluorescence is detected indicating and identifying the presence of a target bacterial species. Wells in which the target bacterial species are not present cannot undergo a phage infection, and thus no marker is produced that can translate into a fluorescent signal. Wells in which the target bacterial species is present in addition to an antibiotic which produces a fluorescent signal are an indication that the target bacterial species is not susceptible or is resistant to the antibiotic. Wells in which the target bacterial species is present in addition to an antibiotic which does not produce a fluorescent signal or produces an attenuated fluorescent signal indicates that the target bacterial species is susceptible to the antibiotic. TABLE 6 below provides an example assay for wells 1-6.

TABLE 6
Example Results of Phage-Based Assay

| | | | | | Emission | |
|---|---|---|---|---|---|---|
| Well | Sample | Phage | Antibiotic | Fluorophore | at 519 nm | Indication |
| 1 | Blood | K1-5::luxAB | None | FAM | Yes | E. coli K1 Present |
| 2 | Blood | K1-5::luxAB | A β-lactam | FAM | Yes | E. coli K1 Resistant |
| 3 | Blood | K1-5::luxAB | A Fluoroquinolone | FAM | No | E. coli K1 Susceptible |
| 4 | Blood | K1-5::luxAB | An Aminoglycoside | FAM | Yes | E. coli K1 Resistant |
| 5 | Blood | K1-5::luxAB | A Tetracycline | FAM | No | E. coli K1 Susceptible |
| 6 | Blood | K1-5::luxAB | A Polymyxin | FAM | No | E. coli K1 Susceptible |

### Analysis for Non-Coding RNA Marker

Alternatively, when the marker codes for unique, non-coding RNA, five unique probes spanning each intron-splice of the non-coding RNA site are designed to harbor fluorophore/quencher pairs that will emit fluorescence at a distinct wavelength. One probe is created with ROX fluorophore and BHQ-2 quencher linked together by a nucleic acid probe corresponding to a sequence spanning the intron splice junction of the non-coding RNA. The nucleic acid linker has at least one RNA base. ROX has a fluorescence emission wavelength of 625 nm.

Spacing between fluorophores and quenchers within the probes are optimized to minimize background fluorescence (<3.4 nm distance) to provide suppression of signal >95%. A single RNA base is positioned within each probe to maximize efficiency of its cleavage by RNase HII. More than one RNA base can be considered, if needed.

The wells of Example 3 are tested for the presence of non-coding RNA using RT-HDA on the SOLANA system marketed by Quidel, Corp. A master mix of enzymes (reverse transcriptase, polymerase, helicase, and RNase HII) is added to cultures to isolate any non-coding RNA present therein. The non-coding RNA is amplified using the enzyme mixture and the marker or its amplicon is detected by addition of the corresponding probe and measuring fluorescence emission at the correct wavelength, e.g., 625 nm for the ROX-based probe. Interpretation of the results is conducted similar to the example provided in TABLE 6 above.

Other embodiments: The preceding examples can be repeated with similar success by substituting the generically or specifically described reactants and/or operating conditions described elsewhere in the specification for those used in the preceding examples.

From the foregoing description, one skilled in the art can easily ascertain the essential characteristics of the methods and, without departing from the spirit and scope thereof, can make various changes and modifications to adapt it to various usages and conditions.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described in the foregoing paragraphs. In addition, the materials, methods, and examples are illustrative only and not intended to be limiting. In case of conflict, the present specification, including definitions, will control.

All United States patents and published or unpublished United States patent applications cited herein are incorporated by reference. All published foreign patents and patent applications cited herein are hereby incorporated by reference. All published references, documents, manuscripts, scientific literature cited herein are hereby incorporated by reference. All identifier and accession numbers pertaining to scientific databases (e.g., NCBI, GENBANK, EBI) that are hereby incorporated by reference.

We claim:

1. A method for simultaneously identifying a bacterial species and determining a susceptibility of the bacterial species to a test antimicrobial agent, comprising,
(a) providing a transforming phage specific to the bacterial species, wherein the transforming phage is an engineered or recombinant phage having a gene for encoding a marker, the gene being heterologous to both humans and the bacterial species;
(b) preparing a first culture by culturing the bacterial species in the absence of the test antimicrobial agent to generate a primary test antimicrobial-free culture and culturing the primary test antimicrobial-free culture in the presence of the transforming phage;
(c) preparing a second culture by culturing the bacterial species in the presence of the test antimicrobial agent to generate a primary test antimicrobial-containing culture; culturing the primary test antimicrobial-containing culture in the presence of the transforming phage; and
(d) analyzing the first and second cultures to determine a level or activity of the marker in each of the first and second cultures.

2. The method of claim 1, wherein a detection of the marker in the first culture provides a positive identification of the bacterial species as the bacterial species to which the transforming phage is specific.

3. The method of claim 1, wherein a reduction in the level or activity of the marker in the second culture compared to the level or activity of the marker in the first culture indicates that the bacterial species is susceptible to the test antimicrobial agent.

4. The method of claim 1, wherein a uniformity or an increase in the level or activity of the marker in the second culture compared to the level or activity of the marker in the first culture indicates that the bacterial species is not susceptible to or is resistant to the test antimicrobial agent.

5. The method of claim 1, wherein the bacterial species is selected from the group consisting of Gram-positive and Gram-negative bacteria.

6. The method of claim 5, wherein the bacteria are selected from the group consisting of Bacillus anthracis, Bacillus subtilis, Bacillus thuringiensis, Escherichia coli, Lactobacillus delbrueckii, Lactobacillus plantarum, Lactococcus lactis, Listeria monocytogenes, Pseudomonas aeruginosa, Pseudomonas syringae, Klebsiella, Salmonella, Shigella, and Staphylococcus aureus.

7. The method of claim 1, wherein the phage is a lytic phage; a temperate or lysogenic phage; or a filamentous phage.

8. The method of claim 7, wherein the phage is a lytic or productive phage selected from the group consisting of T4, T7, T3, and MS2.

9. The method of claim 7, wherein the temperate or lysogenic phage is a λ phage.

10. The method of claim 7, wherein the phage is a filamentous phage selected from the group consisting of f1, fd, and M13.

11. The method of claim 1, wherein the gene is of fungal, plant or insect origin.

12. The method of claim 1, wherein the marker is a hydrolytic enzyme.

13. The method of claim 12, wherein the hydrolytic enzyme is selected from the group consisting of cellulases, cutinases, esterases, lipases, phosphoesterases, restriction endonucleases, and proteases.

14. The method of claim 12, wherein analyzing a culture to determine a level or activity of the hydrolytic enzyme comprises detecting hydrolysis of a substrate specific to the hydrolytic enzyme.

15. The method of claim 14, wherein the substrate is an intramolecularly quenched fluorophore or an intramolecularly quenched chromogen.

16. The method of claim 15, wherein the intramolecularly quenched fluorophore or the intramolecularly quenched chromogen is a compound having the formula:
Q-S-F
wherein,
S comprises a substrate specific to the hydrolytic enzyme;
F is a fluorophore, a chemiluminescent molecule, or a chromogen; and
Q is a quencher;
wherein S forms a link between Q and F, and hydrolysis of S severs the link, thereby reducing a quenching effect of Q upon F.

17. The method of claim 16, wherein F is a chromogen and detecting hydrolysis of the substrate comprises detecting a visual color change or acquiring a colorimetric or spectroscopic reading.

18. The method of claim 16, wherein F is a fluorophore and detecting hydrolysis of the substrate comprises detecting fluorescence or acquiring a fluorometric reading.

19. The method of claim 16, wherein the substrate comprises (i) a nucleic acid sequence and the hydrolytic enzyme is a restriction endonuclease specific to the nucleic acid sequence or (ii) a polypeptide, the hydrolytic enzyme is a protease, and the polypeptide is specific to the protease.

20. The method claim 1, wherein the marker is a non-coding ribonucleic acid (RNA) heterologous to both humans and the target species.

21. The method of claim 20, wherein analyzing a culture to determine a level or activity of the non-coding RNA comprises amplifying the non-coding RNA.

22. The method of claim 21, wherein the non-coding RNA is amplified by Reverse Transcriptase Helicase Dependent Amplification (RT-HDA) whereby a complementary deoxyribonucleic acid (cDNA) amplicon corresponding to the non-coding RNA is generated.

23. A method for the diagnosis of a bacterial disease in a subject in need thereof, comprising,
(a) providing a subject sample comprising a bacterial species;
(b) culturing the subject sample to generate a plurality of primary bacterial cultures;
(c) providing a plurality of transforming phages, each of which is specific to a different bacterial species, wherein each transforming phage is an engineered or recombinant phage having a gene for encoding a unique marker, the gene being heterologous to both humans and the bacterial species;
(d) culturing the primary bacterial cultures of (a) in the presence of a transforming phage of (c) to provide a plurality of secondary bacterial cultures, wherein the transforming phage varies among the secondary bacterial cultures;
(e) analyzing the secondary bacterial cultures to determine the presence or absence of the unique marker in the secondary bacterial cultures;
(f) correlating the detection of the unique marker with a presence of a bacterial species; and
(g) correlating the presence of the bacterial species with the bacterial disease.
